SEQUENCE LISTING (11 sequences)

SEQ ID NO: 1 - protein, 1227 aa, Lachnospiraceae bacterium
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 2 - protein, 1182 aa, Leptotrichia wadei
MYMKITKIDGVSHYKKQDKGILKKKWKDLDERKQREKIEARYNKQIESKIYKEFFRLKNKKRIEKEEDQNIKSLYFFIKELYLNEKNEEWELKNINLEILDDKERVIKGYKFKEDVYFFKEGYKEYYLRILFNNLIEKVQNENREKVRKNKEFLDLKEIFKKYKNRKIDLLLKSINNNKINLEYKKENVNEEIYGINPTNDREMTFYELLKEIIEKKDEQKSILEEKLDNFDITNFLENIEKIFNEETEINIIKGKVLNELREYIKEKEENNSDNKLKQIYNLELKKYIENNFSYKKQKSKSKNGKNDYLYLNFLKKIMFIEEVDEKKEINKEKFKNKINSNFKNLFVQHILDYGKLLYYKENDEYIKNTGQLETKDLEYIKTKETLIRKMAVLVSFAANSYYNLFGRVSGDILGTEVVKSSKTNVIKVGSHIFKEKMLNYFFDFEIFDANKIVEILESISYSIYNVRNGVGHFNKLILGKYKKKDINTNKRIEEDLNNNEEIKGYFIKKRGEIERKVKEKFLSNNLQYYYSKEKIENYFEVYEFEILKRKIPFAPNFKRIIKKGEDLFNNKNNKKYEYFKNFDKNSAEEKKEFLKTRNFLLKELYYNNFYKEFLSKKEEFEKIVLEVKEEKKSRGNINNKKSGVSFQSIDDYDTKINISDYIASIHKKEMERVEKYNEEKQKDTAKYIRDFVEEIFLTGFINYLEKDKRLHFLKEEFSILCNNNNNVVDFNININEEKIKEFLKENDSKTLNLYLFFNMIDSKRISEFRNELVKYKQFTKKRLDEEKEFLGIKIELYETLIEFVILTREKLDTKKSEEIDAWLVDKLYVKDSNEYKEYEEILKLFVDEKILSSKEAPYYATDNKTPILLSNFEKTRKYGTQSFLSEIQSNYKYSKVEKENIEDYNKKEEIEQKKKSNIEKLQDLKVELHKKWEQNKITEKEIEKYNNTTRKINEYNYLKNKEELQNVYLLHEMLSDLLARNVAFFNKWERDFKFIVIAIKQFLRENDKEKVNEFLNPPDNSKGKKVYFSVSKYKNTVENIDGIHKNFMNLIFLNNKFMNRKIDKMNCAIWVYFRNYIAHFLHLHTKNEKISLISQMNLLIKLFSYDKKVQNHILKSTKTLLEKYNIQINFEISNDKNEVFKYKIKNRLYSKKGKMLGKNNKFEILENEFLENVKAMLEYSE

SEQ ID NO: 3 - RNA, 25 nt, Artificial Sequence
  Synthetic
  misc_feature (1)..(1): DBCO- TEG
  misc_feature (25)..(25): Biotin
uuuuuuuuuuuuuuuuuuuuuuuuu

SEQ ID NO: 4 - DNA, 25 bp, Artificial Sequence
  Synthetic
  misc (1)..(1): DBCO-TEG
  misc (25)..(25): Biotin
ttttttttttttttttttttttttt

SEQ ID NO: 5 - RNA, 64 nt, Artificial Sequence
  Synthetic
gauuuagacuaccccaaaaacgaaggggacuaaaacccaaccucuucuguaauuuuuaaacuau

SEQ ID NO: 6 - RNA, 28 nt, Artificial Sequence
  Synthetic
auaguuuaaaaauuacagaagagguugg

SEQ ID NO: 7 - RNA, 28 nt, Artificial Sequence
  Synthetic
cuucuucagguuggacagcuggugcugc

SEQ ID NO: 8 - RNA, 41 nt, Artificial Sequence
  Synthetic
uaauuucuacuaaguguagauaagguuuguguguuuaccug

SEQ ID NO: 9 - DNA, 33 bp, Artificial Sequence
  Synthetic
acctgggggagtattgcggaggaaggtttgtgt

SEQ ID NO: 10 - DNA, 31 bp, Artificial Sequence
  Synthetic
gtttacctgggggagtattgcggaggaaggt

SEQ ID NO: 11 - DNA, 22 bp, Artificial Sequence
  Synthetic
cccaggtaaacacacaaacctt

(19) United States
(12) Patent Application Publication
(10) Pub. No.: US 2024/0150817 A1
(43) Pub. Date: May 9, 2024

(54) CRISPR-MEDIATED CLEAVAGE OF OLIGONUCLEOTIDE-DETECTABLE MARKER CONJUGATES FOR DETECTION OF TARGET ANALYTES

(71) Applicant: NORTHWESTERN UNIVERSITY, Evanston, IL (US)

(72) Inventors: Chad A. Mirkin, Wilmette, IL (US); Devleena Samanta, Evanston, IL (US); Sasha B. Ebrahimi, DeKalb, IL (US)

(21) Appl. No.: 18/283,710

(22) PCT Filed: Mar. 24, 2022
(86) PCT No.: PCT/US22/21786

§ 371 Date: Sep. 22, 2023

Related U.S. Application Data
(60) Provisional application No. 63/165,483, filed on Mar. 24, 2021.

(51) Int. Cl. C12Q 1/6823; C12N 9/22; C12N 15/11; C12Q 1/44; C12Q 1/70

(52) U.S. Cl. CPC C12Q 1/6823; C12N 9/22; C12N 15/11; C12Q 1/44; C12Q 1/70; C12N 2310/16; C12N 2310/20; G01N 2333/922

(57) ABSTRACT

The present disclosure provides a general strategy based on CRISPR and oligonucleotide-detectable marker conjugates that allows sensitive detection of nucleic acid and non-nucleic acid target analytes without stringent temperature requirements. In various aspects, this strategy can be used for rapid and routine detection of viral and bacterial infections, screening of diseases with known biomarkers, and tracking the progression of diseases or response to therapy over time.

### CROSS-REFERENCE TO RELATED APPLICATIONS

This application claims the priority benefit under 35 U.S.C. § 119(e) of U.S. Provisional Patent Application No. 63/165,483, filed Mar. 24, 2021, which is incorporated herein by reference in its entirety.

### STATEMENT OF GOVERNMENT INTEREST

This invention was made with government support under FA8650-15-2-5518 awarded by The Air Force Research Laboratory, FA9550-17-1-0348 awarded by The Air Force Office of Scientific Research, and DE-SC0000989 awarded by The Department of Energy. The government has certain rights in the invention.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “2021-068_Seqlisting.txt”, which was created on Mar. 22, 2022 and is 23,089 bytes in size. The subject matter of the Sequence Listing is incorporated herein in its entirety by reference.

### BACKGROUND

Cas12 and Cas13 proteins in complex with their CRISPR RNA (“CRISPR complex” or “ribonucleoprotein protein (RNP)”) are able to target single-stranded and double stranded DNA or single stranded RNA, respectively. In the case of Cas13, for example, binding of the CRISPR complex to target RNA activates the Cas13, which can then promiscuously cleave RNA in solution. Previous work has shown that if a reporter is present in solution, for example a fluorophore and quencher linked by a short sequence of RNA, an increase in fluorescence can be monitored as a signal for presence of an RNA target of interest [Uttamapinant et al., Nat. Biomed. Eng. 2020, 4, 1140; Ott et al., Cell 2021, 184, 323].

Point-of-care diagnostic platforms can allow for the early detection of various diseases. However, many strategies for sensitive detection such as PCR and ELISA require (i) multistep processes that can only be performed by trained personnel, (ii) specific temperatures for the assay reactions, and (iii) advanced instrumentation for signal readout. In addition, many previous strategies necessitate either target amplification before analysis of samples, or use a fluorescence-based readout, precluding use as at-home diagnostics. Consequently, these techniques typically require centralized facilities with sophisticated infrastructure. Therefore, there is a need for alternative and improved methods of detecting target analytes.

### SUMMARY

The present disclosure provides a general strategy based on CRISPR and oligonucleotide-detectable marker conjugates that allows sensitive detection of nucleic acid and non-nucleic acid target analytes without stringent temperature requirements. In various aspects, this strategy can be used for rapid and routine detection of viral and bacterial infections, screening of diseases with known biomarkers, and tracking the progression of diseases or response to therapy over time.

Applications for the technology provided herein include, but are not limited to, detecting nucleic-acid and non-nucleic acids in solution and in clinical samples (e.g., viral RNA, mRNA, bacterial RNA), and quantifying levels of analytes in solution and complex milieu.

Advantages of the technology disclosed herein include, but are not limited to, specialized equipment is not necessary; no specific temperature requirement (can be performed at room temperature); signal amplification can be achieved without target amplification; small quantities of target analytes can be detected by the naked eye; and the simple workflow allows the technology to be used as a part of at-home testing or point-of-care diagnostics. The present disclosure provides the ability to selectively cleave a reporter in response to a nucleic acid or non-nucleic acid target, and that cleavage is coupled to a signal (e.g., colorimetric, fluorescent, or luminescent readout) that allows the technology to be used as a new point of care diagnostic.

Accordingly, in some aspects the disclosure provides a method of detecting a target analyte in a sample, the method comprising: (A) contacting the sample to a solution comprising: (i) a reporter comprising an oligonucleotide conjugated to a detectable marker, wherein the reporter is immobilized on a surface; (ii) a guide oligonucleotide that hybridizes to (a) the target analyte and/or (b) a nucleic acid sequence partially complementary to an aptamer that becomes available for hybridization to the guide oligonucleotide after the aptamer binds to the target analyte; and (iii) a Cas12 and/or a Cas13 protein that cleaves the reporter after hybridization of the guide oligonucleotide to (a) the target analyte and/or (b) the nucleic acid sequence partially complementary to the aptamer that becomes available for hybridization to the guide oligonucleotide after the aptamer binds to the target analyte, wherein cleavage of the reporter results in release of the detectable marker, wherein the contacting occurs in a vessel; (B) removing the solution comprising the released detectable marker from the vessel, and (C) measuring a signal produced by the released detectable marker in the solution removed from the vessel, wherein the measuring provides for detection of the target analyte in the sample. In further aspects, the disclosure provides a method of detecting a target analyte in a sample, the method comprising: (A) contacting the sample to a solution comprising: (i) a reporter comprising at least one oligonucleotide conjugated to a detectable marker, wherein the reporter is immobilized on a surface; (ii) a guide oligonucleotide that hybridizes to (a) the target analyte and/or (b) a nucleic acid sequence partially complementary to an aptamer that becomes available for hybridization to the guide oligonucleotide after the aptamer binds to the target analyte; and (iii) a Cas12 and/or a Cas13 protein that cleaves the reporter after hybridization of the guide oligonucleotide to (a) the target analyte and/or (b) the nucleic acid sequence partially complementary to the aptamer that becomes available for hybridization to the guide oligonucleotide after the aptamer binds to the target analyte, wherein cleavage of the reporter results in release of the detectable marker, wherein the contacting occurs in a vessel; (B) removing the solution comprising the released detectable marker from the vessel, and (C) measuring a signal produced by the released detectable marker in the solution removed from the vessel, wherein the measuring provides for detection of the target analyte in the sample. In some embodiments, the reporter comprises two or more oligonucleotides conjugated to the detectable marker. In some embodiments, the reporter consists of one oligonucleotide conjugated to one detectable marker. In some embodiments, the Cas12 protein comprises a sequence as set out in SEQ ID NO: 1. In some embodiments, the Cas12 protein comprises a sequence that is at least 80% identical to SEQ ID NO: 1. In some embodiments, the Cas13 protein comprises a sequence as set out in SEQ ID NO: 2. In some embodiments, the Cas13 protein comprises a sequence that is at least 80% identical to SEQ ID NO: 2. In some embodiments, the signal is greater when the target analyte is present in the sample than the signal when the target analyte is not in the sample. In further embodiments, the signal is about 2-fold to 20-fold, 2-fold to 10-fold, 2-fold to 5-fold, 5-fold to 20-fold, or 5-fold to 10-fold greater when the target analyte is present in the sample than the signal when the target analyte is not in the sample. In still further embodiments, the signal is about 1.1-fold, 1.2-fold, 1.3-fold, 1.4-fold, 1.5-fold, 1.6-fold, 1.7-fold, 1.8-fold, 1.9-fold, or 2-fold greater when the target analyte is present in the sample than the signal when the target analyte is not in the sample. In various embodiments, the guide oligonucleotide is RNA or a DNA-RNA chimera. In some embodiments, the oligonucleotide portion of the reporter is DNA, RNA, a DNA-RNA chimera, modified forms thereof, or a combination thereof. In some embodiments, the oligonucleotide of the reporter is DNA, RNA, a DNA-RNA chimera, modified forms thereof, or a combination thereof. In some embodiments, the target analyte is a nucleic acid, a protein, a small molecule, an ion, a carbohydrate, a cell, or a combination thereof. In further embodiments, the ion is a metal ion. In still further embodiments, the metal ion is a mercury ion, a copper ion, a silver ion, a zinc ion, a gold ion, a manganese ion, or a combination thereof. In yet additional embodiments, the ion is a hydrogen ion. In some embodiments, the nucleic acid is a viral nucleic acid. In further embodiments, the viral nucleic acid is from a DNA virus, a RNA virus, or a combination thereof. In still further embodiments, the viral nucleic acid is from a Coronaviridae virus, an Arteriviridae virus, a Roniviridae virus, a Picornaviridae virus, or a combination thereof. In yet additional embodiments, the virus is Coronavirus, MERS, alphacoronavirus HCoV-NL63, betacoronaviruses HCoV-OC43, H1N1 influenza A, influenza BSARS, a variant thereof, or a combination thereof. In further embodiments, the Coronavirus is SARS-CoV-2 and/or a variant thereof. In some embodiments, the nucleic acid is bacterial nucleic acid. In further embodiments, the bacterial nucleic acid is from Myobacterium tuberculosis, E. coli, Staphylococcus aureus, Shigella dysenteriae, or a combination thereof. In some embodiments, the nucleic acid is protozoan nucleic acid. In further embodiments, the protozoan nucleic acid is from Plasmodium falciparum, Plasmodium vivax, Plasmodium ovale, or Plasmodium malariae, or a combination thereof. In some embodiments, the nucleic acid is cancer-related nucleic acid. In further embodiments, the cancer-related nucleic acid is mRNA, miRNA, circulating DNA, or a combination thereof. In still further embodiments, the cancer-related nucleic acid is BRAF, PIK3CA, MGMT, KRAS, TP53, ESR1, EML4-ALK fusion, miR-125b-5p, miR-155, or a combination thereof. In some embodiments, the protein is prostate-specific antigen (PSA) or thrombin. In various embodiments, the small molecule is adenosine triphosphate (ATP), dehydroepiandrosterone (DHEA), dehydroepiandrosterone sulfate (DHEA-S), or a combination thereof. In some embodiments, the oligonucleotide portion of the reporter is about 2 to about 50 nucleotides in length. In some embodiments, the oligonucleotide of the reporter is about 2 to about 50 nucleotides in length. In some embodiments, the guide oligonucleotide is about 10 to about 100 nucleotides in length. In some embodiments, the detectable marker is an enzyme or a catalyst. In various embodiments, the surface is a tube, a bead, a multiwell plate, a hydrogel or a nanoparticle. In further embodiments, the nanoparticle is magnetic. In some embodiments, the vessel is a tube, or a multiwell plate.

### BRIEF DESCRIPTION OF THE DRAWINGS

FIG. 1 shows a schematic of an exemplary method of the disclosure.

FIG. 2 shows results of the experiment described in Example 1.

FIG. 3 shows (A) Conventional CRISPR-Cas13 sensing scheme. A CRISPR-Cas13 RNP is used whose gRNA has complementarity to a target RNA of interest. Upon target binding, Cas13 is activated and cleaves a fluorophore-quencher labeled ssRNA reporter. This separates the fluorophore and quencher, thereby turning on fluorescence. (B) Dual signal amplification scheme for RNA detection using a probe set consisting of CRISPR-Cas13 and HRP. First, HRP conjugated with biotinylated ssRNA is bound to the surface of streptavidin modified microbeads. The microbeads are then added to a solution containing a CRISPR-Cas13 RNP whose gRNA has complementarity to a target RNA of interest. Upon target binding, Cas13 is activated and cleaves the ssRNA bound to the microbead surface, thereby releasing HRP into solution. (C) HRP released into solution can be detected colorimetrically using TMB substrate that is oxidized in the presence of HRP to yield a blue signal (D) Schematic to scale showing the entry of the Cas enzyme to cleave the ssRNA-modified HRP from the microbead surface.

FIG. 4 shows A) UV-Vis spectrum of HRP-DNA conjugates. 1.47 DNA strands per HRP were calculated. B) UV-Vis spectrum of HRP-RNA conjugates. 1.66 DNA strands per HRP were calculated.

FIG. 5 shows results of experiments using the CRISPR-Cas13/HRP probe set for measuring a synthetic RNA target for SARS-CoV-2. On the y-axis, I_{f}/I₀ is the enhancement factor, where I_{f} and I₀ are the signal intensities in the presence and absence of the target, respectively. I_{c,t} denotes signal intensity after target addition at a concentration of c at time, t. I_{0,0} denotes signal intensity in the absence of target at the initial timepoint. Panels A, B, C, and F use a colorimetric substrate for HRP, panel D uses a fluorescent substrate for HRP, and panel E uses a luminescent substrate for HRP (A) Colorimetric enhancement factor over time showing approximately 25 fold signal enhancement at 120 minutes (B) A calibration curve for colorimetric response at x min yielding a LOD of 400 fM (C). Visual detection of 1 pM target at x min (D) A calibration curve for fluorescence response at x min yielding an enhancement factor of 25 and a LOD of 10 fM (E) A calibration curve for luminescence response at x min yielding an enhancement factor of 75 and a LOD of 10 fM (F) Challenging the probe with an off-target scramble sequence showing that the detector is selective.

FIG. 6 shows visual detection of ORF1ab target RNA at varying concentrations. Reading taken after 35 minutes.

FIG. 7 depicts the fluorescence kinetics of ORF1ab RNA target sensing using a dual amplification fluorometric method as described in Example 2.

FIG. 8 shows fluorescence kinetics of calibration curve for ORF1ab RNA target detection using fluorophore-quencher reporter RNA.

FIG. 9 shows A) Fluorescence enhancement after 20 minutes using fluorophore-quencher reporter RNA. B) Fluorescence enhancement after 20 minutes using fluorogenic HRP substrate shows approximately 30 times increased sensitivity compared to fluorophore-quencher reporter system.

FIG. 10 shows detection of full SARS-CoV-2 RNA transcript using a dual amplification colorimetric method as described in Example 2.

FIG. 11 shows results of experiments using the CRISPR-Cas12/HRP probe set for detecting a non-nucleic acid target (ATP). (A) and (B) A schematic of the sensing strategy. In (A), the complement (activator) to the gRNA is blocked by two aptamers for ATP. Upon ATP binding to its aptamers, the activator becomes free. In (B), HRP conjugated with biotinylated ssDNA is bound to the surface of streptavidin modified microbeads. The microbeads are then added to a solution containing a Crispr-Cas12 RNP and the solution from part (A). When sufficient amounts of free activator are present, Cas12 is activated and cleaves the ssDNA bound to the microbead surface, thereby releasing HRP into solution. (C) A calibration curve for colorimetric response at x min yielding an enhancement factor of 20 and a LOD of 0.2 μM (D) Challenging the probe with structurally similar nucleoside triphosphate molecules showing that the detector was selective for ATP (E) ATP sensing in human serum samples with clear detection at concentrations as low as 1 μM (F) Challenging the probe with an off-target scramble sequence showing that the detector was selective.

FIG. 12 shows fluorescence enhancement over time for detection of ATP using fluorophore-quencher reporter DNA.

FIG. 13 shows signal enhancement over time for colorimetric ATP detection using a dual amplification method as described in Example 2.

FIG. 14 depicts a positive control for HRP-RNA cleavage from surface using RNase A.

### DETAILED DESCRIPTION

### Terminology

All language such as “from,” “to,” “up to,” “at least,” “greater than,” “less than,” and the like include the number recited and refer to ranges which can subsequently be broken down into sub-ranges.

A range includes each individual member. Thus, for example, a group having 1-3 members refers to groups having 1, 2, or 3 members. Similarly, a group having 6 members refers to groups having 1, 2, 3, 4, or 6 members, and so forth.

As used in this specification and the appended claims, the articles “a” and “an” refer to one or to more than one (for example, to at least one) of the grammatical object of the article.

“About” and “approximately” shall generally mean an acceptable degree of error for the quantity measured given the nature or precision of the measurements. Exemplary degrees of error are within 20-25 percent (%), for example, within 20 percent, 10 percent, 5 percent, 4 percent, 3 percent, 2 percent, or 1 percent of the stated value or range of values.

The terms “polynucleotide” and “oligonucleotide” are interchangeable as used herein.

A “reporter” as used herein is an oligonucleotide that is conjugated to a detectable marker. In various embodiments, the oligonucleotide portion of the reporter is DNA, RNA, a DNA-RNA chimera, modified forms thereof, or a combination thereof.

A “CRISPR complex” as used herein refers to a guide oligonucleotide that is associated with a Cas12 or Cas13 protein. A “CRISPR complex” may also be referred to as a ribonucleotide protein (RNP).

A “subject” is a vertebrate organism. The subject can be a non-human mammal (e.g., a mouse, a rat, or a non-human primate), or the subject can be a human subject.

A “sample” as used herein generally refers to a sample from a subject. Samples contemplated by the disclosure include, without limitation, saliva, sputum, blood, mucous, a swab from skin or a mucosal membrane, urine, a cell lysate, or a combination thereof.

All references, patents, and patent applications disclosed herein are incorporated by reference with respect to the subject matter for which each is cited, which in some cases may encompass the entirety of the document.

### Methods

The present disclosure is generally directed to methods of detecting a target analyte. In any of the aspects or embodiments of the disclosure, the target analyte is in a sample. Methods of the disclosure utilize the properties of the CRISPR-Cas (clustered regularly interspaced short palindromic repeats-CRISPR associated) proteins Cas12 and Cas13. In some embodiments, the Cas12 protein comprises or consists of a sequence as set out in SEQ ID NO: 1, or a sequence that is or is at least about 80%, 85%, 90%, 95%, 99%, or 100% identical to SEQ ID NO: 1. In some embodiments, the Cas13 protein comprises or consists of a sequence as set out in SEQ ID NO: 2, or a sequence that is or is at least about 80%, 85%, 90%, 95%, 99%, or 100% identical to SEQ ID NO: 2. Cas12 possesses the ability to indiscriminately cleave single-stranded DNA (such as a DNA oligonucleotide portion of a reporter as described herein) once activated by a target DNA molecule matching its associated guide oligonucleotide. Thus, Cas12 is useful for detecting small amounts of target DNA in a sample. On the other hand, Cas13 targets RNA, not DNA. Upon being activated by a target analyte that is RNA (e.g., single-stranded RNA) that has sufficient complementarity to its associated guide oligonucleotide to hybridize, it activates a nonspecific RNase activity and cleaves nearby RNA (such as a RNA oligonucleotide portion of a reporter as described herein) irrespective of the sequence of the nearby RNA. In some embodiments, Cas12 is used for RNA detection and/or Cas13 is used for DNA detection. In these embodiments, the methods comprise a step of converting the target analyte from DNA to RNA (for Cas13) or from RNA to DNA (for Cas12). Thus, Cas12 and Cas13 can each be used to detect RNA and/or DNA in a sample.

Reporters are cleaved when a Cas protein reaches the surface to which the reporter is immobilized (i.e., the Cas protein generally needs to diffuse through the solution to reach the surface to which the reporter is immobilized). Thus, in some embodiments, if a flat surface like a plate is used, only Cas protein near the surface of the plate is able to access the surface in a reasonable amount of time. However, depending on the target concentration one wishes to detect, a plate may be preferable due to its ease of use. For example and without limitation, while a plate may increase the limit of detection, for some applications that limit of detection will be appropriate. In some embodiments, the disclosure provides beads (to which one or a plurality of reporters is immobilized) which can be homogeneously dispersed throughout the solution to mitigate potential diffusion limitations. Further, because reporters of the disclosure are immobilized to a surface, an important consideration is the nucleotide length of the oligonucleotide portion of the reporter. In general, if the nucleotide length is too short, a Cas protein will not be able to efficiently access the oligonucleotide portion of the reporter and cleave it. On the other hand, if the oligonucleotide portion of the reporter is too long, the Cas protein might cleave the same reporter at multiple sites as opposed to different reporters, thereby decreasing the signal to noise ratio. Further, in any of the aspects or embodiments of the disclosure, a plurality of reporters is used. In some embodiments, each reporter in the plurality contains approximately one oligonucleotide conjugated to one detectable marker. In some embodiments, a plurality of reporters is used, wherein each reporter consists of one oligonucleotide conjugated to one detectable marker. In some embodiments, for every detectable marker there is exactly one oligonucleotide conjugated thereto so that cleavage of the oligonucleotide portion of the reporter by a CRISPR complex results in stoichiometric amounts of detectable marker being released. In some embodiments, a plurality of reporters is used, wherein each reporter comprises more than one oligonucleotide conjugated to one detectable marker. In further embodiments, a plurality of reporters is used, wherein each reporter comprises or consists of about 1, 2, 3, 4, 5, 6, 7, 8, 9, 10, or more oligonucleotides conjugated to one detectable marker. In some embodiments, the target is a relatively high abundance target.

A “guide oligonucleotide” as used herein refers to an oligonucleotide having sufficient complementarity to a target analyte (and/or a nucleic acid sequence partially complementary to an aptamer that becomes available for hybridization to the guide oligonucleotide after the aptamer binds to the target analyte) to associate with the target analyte and to promote binding of a CRISPR complex comprising the guide oligonucleotide and the Cas12 or Cas13 protein to the target analyte. In various embodiments, the guide oligonucleotide is 100% complementary to the target analyte (and/or a nucleic acid sequence partially complementary to an aptamer that becomes available for hybridization to the guide oligonucleotide after the aptamer binds to the target analyte), i.e., a perfect match, while in other aspects, the guide oligonucleotide is at least about 95% complementary to the target analyte over the length of the guide oligonucleotide, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, or at least about 20% complementary to the target analyte (and/or a nucleic acid sequence partially complementary to an aptamer that becomes available for hybridization to the guide oligonucleotide after the aptamer binds to the target analyte) over the length of the guide oligonucleotide. In general, a guide oligonucleotide is between about 10 to about 100 nucleotides in length. In any of the aspects or embodiments of the disclosure, the guide oligonucleotide is RNA (guide RNA, or gRNA). In any of the aspects or embodiments of the disclosure, the guide oligonucleotide is single-stranded RNA. In some embodiments, the guide oligonucleotide is a DNA-RNA chimera (see, e.g., Kim et al., Nucleic Acids Research 48(15): 8601-8616 (2020). In various embodiments, the guide oligonucleotide is about 10 to about 100 nucleotides in length. More specifically, a guide oligonucleotide is about 10 to about 90 nucleotides in length, about 10 to about 80 nucleotides in length, about 10 to about 70 nucleotides in length, about 10 to about 60 nucleotides in length, about 10 to about 50 nucleotides in length about 10 to about 45 nucleotides in length, about 10 to about 40 nucleotides in length, about 10 to about 35 nucleotides in length, about 10 to about 30 nucleotides in length, about 10 to about 25 nucleotides in length, about 10 to about 20 nucleotides in length, about 10 to about 15 nucleotides in length, and all oligonucleotides intermediate in length of the sizes specifically disclosed to the extent that the oligonucleotide is able to achieve the desired result. Accordingly, in various embodiments, a guide oligonucleotide is or is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more nucleotides in length. In further embodiments, a guide oligonucleotide is less than 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more nucleotides in length. In some embodiments, the target analyte is a nucleic acid and the guide oligonucleotide comprises a nucleotide sequence that is sufficiently complementary to the target nucleic acid to hybridize to the target nucleic acid under the conditions being used. Thus, in this way the guide oligonucleotide directly associates with the target analyte. Accordingly, in some aspects, the disclosure contemplates that a guide oligonucleotide hybridizes to a target nucleic acid. When the target analyte is a nucleic acid, the target nucleic acid is also referred to herein as an “initiator” nucleic acid. Following hybridization of the guide oligonucleotide to the target nucleic acid, the Cas12 or Cas13 protein that is complexed with the guide oligonucleotide becomes activated and indiscriminately cleaves DNA (in the case of Cas12) or RNA (in the case of Cas13) oligonucleotides, such as the oligonucleotide portions of a reporter as described herein. Cleavage of the oligonucleotide portions of a reporter results in release of the detectable marker from the oligonucleotide portions of the reporter. In various embodiments, and as described herein, the oligonucleotide portion of the reporter is DNA, RNA, a DNA-RNA chimera, modified forms of any of the foregoing, or a combination thereof. The released detectable marker is then recovered, for example, by removal of the solution comprising the released detectable marker from the vessel in which the assay was performed and measuring a signal produced by the released detectable marker in the solution, wherein the measuring provides for detection of the target in the sample. In the context of measuring a signal produced by a detectable marker, any method of measuring the signal is contemplated by the disclosure. For example and without limitation, in various embodiments the measuring is performed by naked eye (based on, e.g., a color change), and/or the measuring is performed by an instrument capable of detecting, e.g., a fluorescent, luminescent, and/or colorimetric signal. In general, an increase in the signal produced by the detectable marker compared to a control signal when the target analyte is not in the sample is indicative of presence of the target analyte in the sample. Moreover, in any of the aspects or embodiments of the disclosure, the magnitude of the signal produced by the released detectable marker is proportional to the amount of the target analyte in the sample. In various embodiments, the signal produced by the released detectable marker is at least two-fold greater when the target is present in the sample than the signal when the target is not in the sample. In further embodiments, the signal produced by the released detectable marker is about 1.1-fold, 1.2-fold, 1.3-fold, 1.4-fold, 1.5-fold, 1.6-fold, 1.7-fold, 1.8-fold, 1.9-fold, 2-fold to 20-fold, 2-fold to 10-fold, 2-fold to 5-fold, 5-fold to 20-fold, or 5-fold to 10-fold greater when the target is present in the sample than the signal when the target is not in the sample. In general, any fold-increase that is statistically different from the signal obtained when the target is not in the sample is contemplated by the disclosure.

In some embodiments, the target analyte is a non-nucleic acid (e.g., a protein, a small molecule, a carbohydrate). Such non-nucleic acid target analytes may be detected, for example and without limitation, via an aptamer or a DNAzyme. In general, aptamers are nucleic acid or peptide binding species capable of tightly binding to and discreetly distinguishing target ligands [Yan et al., RNA Biol. 6(3) 316-320 (2009), incorporated by reference herein in its entirety]. Aptamers, in some embodiments, may be obtained by a technique called the systematic evolution of ligands by exponential enrichment (SELEX) process [Tuerk et al., Science 249:505-10 (1990), U.S. Pat. Nos. 5,270,163, and 5,637,459, each of which is incorporated herein by reference in their entirety]. General discussions of nucleic acid aptamers are found in, for example and without limitation, Nucleic Acid and Peptide Aptamers: Methods and Protocols (Edited by Mayer, Humana Press, 2009) and Crawford et al., Briefings in Functional Genomics and Proteomics 2(1): 72-79 (2003). Additional discussion of aptamers, including but not limited to selection of RNA aptamers, selection of DNA aptamers, selection of aptamers capable of covalently linking to a target protein, use of modified aptamer libraries, and the use of aptamers as a diagnostic agent and a therapeutic agent is provided in Kopylov et al., Molecular Biology 34(6): 940-954 (2000) translated from Molekulyarnaya Biologiya, Vol. 34, No. 6, 2000, pp. 1097-1113, which is incorporated herein by reference in its entirety. In various aspects, an aptamer is between 10-100 nucleotides in length. In various embodiments, aptamers may be single stranded, double stranded, or partially double stranded. Aptamers can undergo a conformational change upon binding to a target analyte, thereby exposing a nucleic acid sequence partially complementary to the aptamer and making it available for hybridization to a guide oligonucleotide. In the absence of the target analyte, the conformational change does not occur or occurs to a lesser extent; thus, the nucleic acid sequence partially complementary to the aptamer to which the guide oligonucleotide can hybridize is not exposed. For example, in some embodiments the aptamer comprises a nucleic acid sequence that hybridizes to another portion of the aptamer in the absence of the target analyte, and binding of the aptamer to a target analyte results in dehybridization of the nucleic acid sequence, thereby making the nucleic acid sequence available for hybridization to a guide oligonucleotide. Accordingly, in some aspects, the disclosure contemplates that a guide oligonucleotide hybridizes to a nucleic acid sequence partially complementary to an aptamer that becomes available for hybridization to the guide oligonucleotide after the aptamer binds to the target analyte. Following hybridization of the guide oligonucleotide to the nucleic acid sequence partially complementary to an aptamer that becomes available for hybridization to the guide oligonucleotide after the aptamer binds to the target analyte, the Cas12 or Cas13 protein that is complexed with the guide oligonucleotide becomes activated and will indiscriminately cleave DNA (in the case of Cas12) or RNA (in the case of Cas13) oligonucleotides, such as the oligonucleotide portions of a reporter as described herein. Cleavage of the oligonucleotide portions of a reporter results in release of the detectable marker from the oligonucleotide portions of the reporter. As described herein, in various embodiments the oligonucleotide portion of the reporter is DNA, RNA, a DNA-RNA chimera, modified forms thereof, or a combination thereof. The released detectable marker is then recovered, for example, by removal of the solution comprising the released detectable marker from the vessel in which the assay was performed, and a signal produced by the released detectable marker in the solution is measured, wherein the measuring provides for detection of the target in the sample. In general, an increase in the signal produced by the detectable marker compared to a control signal when the target analyte is not in the sample is indicative of presence of the target analyte in the sample. Moreover, in any of the aspects or embodiments of the disclosure, the magnitude of the signal produced by the released detectable marker is proportional to the amount of the target analyte in the sample. In various embodiments, the signal produced by the released detectable marker is at least two-fold greater when the target is present in the sample than the signal when the target is not in the sample. In further embodiments, the signal produced by the released detectable marker is or is at least about 1.1-fold, 1.2-fold, 1.3-fold, 1.4-fold, 1.5-fold, 1.6-fold, 1.7-fold, 1.8-fold, 1.9-fold, 2-fold, 5-fold, 10-fold, 20-fold, 2-fold to 20-fold, 2-fold to 10-fold, 2-fold to 5-fold, 5-fold to 20-fold, or 5-fold to 10-fold greater when the target is present in the sample than the signal when the target is not in the sample. In general, any fold-increase that is statistically different from the signal obtained when the target is not in the sample is contemplated by the disclosure.

In some aspects, the disclosure provides methods of detecting more than one target analyte in a sample. Accordingly, in some embodiments, a sample is contacted with a solution comprising more than one reporter and more than one CRISPR complex. In some embodiments, the solution comprises (i) a first reporter comprising a DNA oligonucleotide conjugated to a first detectable marker; (ii) a second reporter comprising a RNA oligonucleotide conjugated to a second detectable marker, wherein the first reporter and the second reporter are immobilized on a surface; (iii) a first CRISPR complex comprising a Cas12 protein and a first guide oligonucleotide having sufficient complementarity to hybridize to (a) a first target analyte or (b) a nucleic acid sequence partially complementary to an aptamer that becomes available for hybridization to the first guide oligonucleotide after the aptamer binds to the first target analyte; (iv) a second CRISPR complex comprising a Cas13 protein and a second guide oligonucleotide having sufficient complementarity to hybridize to (a) a second target analyte or (b) a nucleic acid sequence partially complementary to an aptamer that becomes available for hybridization to the second guide oligonucleotide after the aptamer binds to the second target analyte. If the sample comprises the first target analyte, then the contact will result in cleavage of the first reporter and release of the first detectable marker. If the sample comprises the second target analyte, then the contact will result in cleavage of the second reporter and release of the second detectable marker. If the sample comprises both the first target analyte and the second target analyte, then the contact will result in (i) cleavage of the first reporter and release of the first detectable marker, and (ii) cleavage of the second reporter and release of the second detectable marker. Assaying the solution that is removed after the contacting for the presence of the first detectable marker and the second detectable marker provides for the detection of the first target analyte, the second target analyte, or both in the sample. In some embodiments, the guide oligonucleotide is a guide RNA. In various embodiments, the first reporter and/or the second reporter comprises a modified oligonucleotide.

In some embodiments, the first detectable marker and the second detectable marker are different.

In any of the aspects or embodiments of the disclosure, a method as described herein is performed entirely at room temperature (e.g., about 20° C. to about 25° C.). In any of the aspects or embodiments of the disclosure, the target analyte is not amplified prior to cleavage of the reporter.

In various aspects, a vessel is a tube or a multiwall plate. In various embodiments, the surface is a tube, a bead, a hydrogel, a multiwell plate, or a nanoparticle. Thus, in some embodiments, a tube is the vessel in which the assay is performed and also provides the surface to which the reporter is immobilized. In some embodiments, the surface is a nanoparticle and the reporter is attached to the surface of the nanoparticle. In some embodiments, the surface is a microbead and the reporter is attached to the surface of the microbead. In further embodiments, the nanoparticle is magnetic, such that a magnetic field may be applied to the tube following contact of a sample to a solution comprising a reporter, a guide oligonucleotide, and a Cas12 or Cas13 protein, each as described herein. In further embodiments, the microbead is magnetic, such that a magnetic field may be applied to the tube following contact of a sample to a solution comprising a reporter, a guide oligonucleotide, and a Cas12 or Cas13 protein, each as described herein. Thus, when the solution is removed from the vessel to measure any released detectable marker, application of the magnetic field before the solution is removed inhibits removal of the nanoparticles.

In various embodiments, the oligonucleotide portion of a reporter is about 2 to about 50 nucleotides in length, or about 10 to about 50 nucleotides in length. More specifically, an oligonucleotide portion of a reporter is about 2 to about 45 nucleotides in length, about 2 to about 40 nucleotides in length, about 2 to about 35 nucleotides in length, about 2 to about 30 nucleotides in length, about 2 to about 25 nucleotides in length, about 2 to about 20 nucleotides in length, about 2 to about 15 nucleotides in length, or about 2 to about 10 nucleotides in length, or about 2 to about 5 nucleotides in length. In further embodiments, an oligonucleotide portion of a reporter is about 10 to about 50 nucleotides in length about 10 to about 45 nucleotides in length, about 10 to about 40 nucleotides in length, about 10 to about 35 nucleotides in length, about 10 to about 30 nucleotides in length, about 10 to about 25 nucleotides in length, about 10 to about 20 nucleotides in length, about 10 to about 15 nucleotides in length, and all oligonucleotides intermediate in length of the sizes specifically disclosed to the extent that the oligonucleotide is able to achieve the desired result. Accordingly, in various embodiments, an oligonucleotide portion of a reporter is or is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more nucleotides in length. In further embodiments, an oligonucleotide portion of a reporter is less than 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more nucleotides in length.

In any of the aspects or embodiments of the disclosure, an assay (e.g., polymerase chain reaction (PCR)) is performed prior to the methods described herein (e.g., prior to cleavage of the reporter) in order to amplify a target analyte.

### Target Analytes

As described herein, and in various embodiments, the target analyte is a nucleic acid, a protein, a small molecule, an ion, a carbohydrate, a cell, or a combination thereof. Thus, target analytes of the disclosure also include, in some embodiments, non-nucleic acids. In various embodiments, the non-nucleic acid target is a protein, a small molecule, a carbohydrate, an ion, a cell, or a combination thereof. In various embodiments, the protein is prostate-specific antigen (PSA) or thrombin. In various embodiments, the cell is a cancer cell. In some embodiments, the ion is a metal ion. In further embodiments, the metal ion is a mercury ion, a copper ion, a silver ion, a zinc ion, a gold ion, a manganese ion, or a combination thereof. In some embodiments, the ion is a hydrogen ion. In some embodiments, the nucleic acid is from a microbial pathogen. In some embodiments, the microbe is a virus, a bacterium, a fungus, or a parasite. In some embodiments, the nucleic acid is a viral nucleic acid, and in further embodiments the viral nucleic acid is from a DNA virus, a RNA virus, or a combination thereof. In some embodiments, the viral nucleic acid is from a Coronaviridae virus, an Arteriviridae virus, a Roniviridae virus, a Picornaviridae virus, or a combination thereof. In some embodiments, the virus is Coronavirus, MERS, alphacoronavirus HCoV-NL63, betacoronaviruses HCoV-OC43, H1N1 influenza A, influenza BSARS, or a combination thereof. In some embodiments, the Coronavirus is SARS-CoV-2 and/or a variant thereof. In some embodiments, the nucleic acid is bacterial nucleic acid. In further embodiments, the bacterial nucleic acid is from Myobacterium tuberculosis, E. coli, Staphylococcus aureus, Shigella dysenteriae, or a combination thereof. In some embodiments, the nucleic acid is protozoan nucleic acid. In further embodiments, the protozoan nucleic acid is from Plasmodium falciparum, Plasmodium vivax, Plasmodium ovale, or Plasmodium malariae, or a combination thereof. In some embodiments, the nucleic acid is a cancer-related nucleic acid. In further embodiments, the cancer-related nucleic acid is mRNA, miRNA, circulating DNA, or a combination thereof. In further embodiments, the cancer-related nucleic acid is BRAF, PIK3CA, MGMT, KRAS, TP53, ESR1, EML4-ALK fusion, miR-125b-5p, miR-155, or a combination thereof.

The term “small molecule,” as used herein, refers to a chemical compound, or any other low molecular weight organic compound, either natural or synthetic. By “low molecular weight” is meant compounds having a molecular weight of less than 1500 Daltons, typically between 100 and 700 Daltons. In various embodiments, the small molecule is adenosine triphosphate (ATP), dehydroepiandrosterone (DHEA), dehydroepiandrosterone sulfate (DHEA-S), or a combination thereof.

In any of the aspects or embodiments of the disclosure, a target analyte is a nucleic acid that comprises a nucleotide sequence to which a guide oligonucleotide is sufficiently complementary, such that hybridization between the target analyte and the guide oligonucleotide promotes binding of a CRISPR complex comprising the guide oligonucleotide and the Cas12 or Cas13 protein to the target analyte. In some embodiments, the guide oligonucleotide is a guide RNA. Nucleic acids contemplated by the disclosure to be target analytes include RNA oligonucleotides, DNA oligonucleotides, or a combination thereof. The target RNA oligonucleotides and DNA oligonucleotides are, in various embodiments, single stranded, double stranded, partially double stranded, or a combination thereof.

In some aspects, the target analyte is a non-nucleic acid that is recognized and bound by an aptamer, wherein aptamer binding to the non-nucleic acid results in a nucleic acid sequence partially complementary to the aptamer becoming available for hybridization to a guide oligonucleotide. In some embodiments, the guide oligonucleotide is a guide RNA. In some embodiments, the target analyte is ATP. Hybridization of the guide oligonucleotide to the nucleic acid sequence partially complementary to the aptamer that becomes available when the aptamer binds to the non-nucleic acid promotes binding of a CRISPR complex comprising the guide oligonucleotide and the Cas12 or Cas13 protein to the non-nucleic acid.

### Reporter

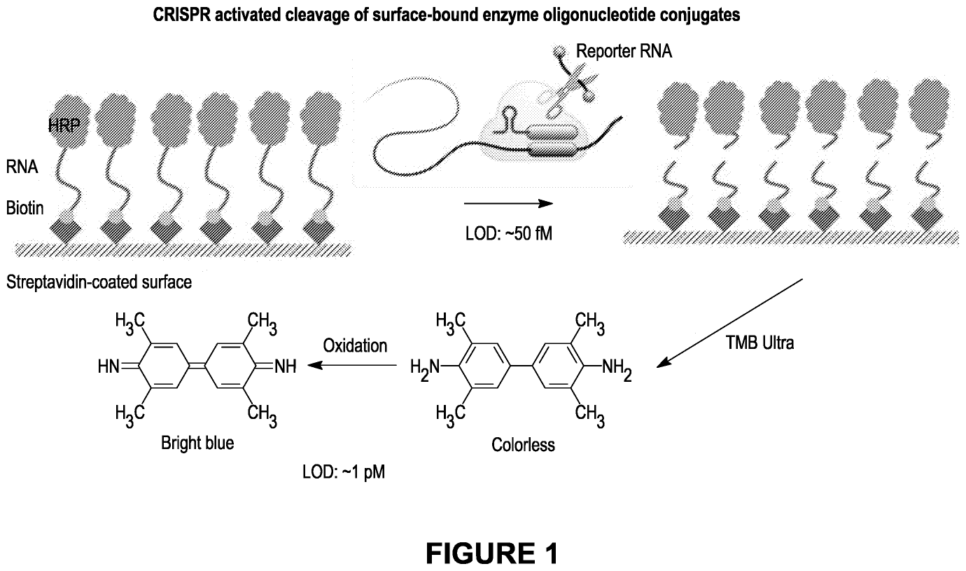

A “reporter” as used herein is an oligonucleotide that is conjugated to a detectable marker. In some embodiments, the reporter comprises about one oligonucleotide conjugated to one detectable marker. In some embodiments, the reporter consists of one oligonucleotide conjugated to one detectable marker. In further embodiments, the reporter comprises or consists of about or at least about 1, 2, 3, 4, 5, 6, 7, 8, 9, 10, or more oligonucleotides conjugated to one detectable marker. In still further embodiments, the reporter comprises or consists of less than about 2, 3, 4, 5, 6, 7, 8, 9, or 10 oligonucleotides conjugated to one detectable marker. In various embodiments, the oligonucleotide portion of the reporter is DNA, RNA, a DNA-RNA chimera, modified forms thereof, or a combination thereof. Reporters of the disclosure are immobilized on a surface via any means (for example and without limitation, via a biotin-streptavidin linkage). In any of the aspects or embodiments of the disclosure, one end of the oligonucleotide portion of the reporter is attached to the surface and the other end of the reporter comprises the detectable marker. The oligonucleotide portion of the reporter may be attached to the surface via its 5′ or 3′ terminus. The detectable marker is conjugated to the terminus of the oligonucleotide portion of the reporter that is not attached to the surface. See, e.g., FIG. 1.

Any method of attaching an oligonucleotide to a surface, and of attaching a detectable marker to an oligonucleotide, may be used according to the disclosure. For example and without limitation, in some embodiments one terminus of the oligonucleotide portion of the reporter is conjugated to biotin and the opposite terminus of the oligonucleotide portion of the reporter is conjugated to the detectable marker. The surface is coated with streptavidin, such that binding of the biotin to the streptavidin results in immobilization of the reporter to the surface.

Detectable markers contemplated for use according to the disclosure include any marker that produces no substantial signal until the released detectable marker is removed from the vessel and measured. Detectable markers contemplated by the disclosure include enzymes (e.g., horseradish peroxidase, alkaline phosphatase, β-galactosidase, glucose oxidase, catalase), catalysts, or a combination thereof. In some embodiments, the detectable marker is an oligonucleotide modified with a fluorophore that is cleaved off the surface. In such embodiments, fluorescence of what was cleaved off the surface is measured as the signal. In further embodiments, the detectable marker is an oligonucleotide modified particle (e.g., fluorescent quantum dots) having a detectable signal that is cleaved off the surface and measured.

### Kits

In some aspects, the disclosure also provides kits comprising a vessel comprising an immobilized reporter comprising an oligonucleotide conjugated to a detectable marker; a guide oligonucleotide that hybridizes to (a) a target analyte and/or (b) a nucleic acid sequence partially complementary to an aptamer that becomes available for hybridization to the guide oligonucleotide after the aptamer binds to a target analyte; and a Cas12 and/or Cas13 protein. In some embodiments, the contents of the vessel are in a solution. In some embodiments, the vessel is a tube. In some embodiments, the reporter is immobilized to the surface inside the tube. In some embodiments, the reporter is immobilized to a nanoparticle that is inside the vessel. In some embodiments, the guide oligonucleotide is a guide RNA. In some embodiments, the guide oligonucleotide is associated with a Cas12 or Cas13 protein in a CRISPR complex. In some embodiments, the kit comprises a second vessel comprising an immobilized reporter comprising an oligonucleotide conjugated to a detectable marker; a guide oligonucleotide that hybridizes to (a) a target analyte and/or (b) a nucleic acid sequence partially complementary to an aptamer that becomes available for hybridization to the guide oligonucleotide after the aptamer binds to a target analyte; and a Cas12 and/or Cas13 protein. In various embodiments, the vessel and the second vessel are used to detect the same target analyte. In further embodiments, the vessel and the second vessel are used to detect different target analytes.

In some embodiments, the disclosure provides a kit comprising a vessel comprising more than one reporter and more than one CRISPR complex. In some embodiments, the vessel comprises (i) a first reporter comprising a DNA oligonucleotide conjugated to a first detectable marker; (ii) a second reporter comprising a RNA oligonucleotide conjugated to a second detectable marker, wherein the first reporter and the second reporter are immobilized on a surface; (iii) a first CRISPR complex comprising a Cas12 protein and a first guide oligonucleotide having sufficient complementarity to hybridize to (a) a first target analyte or (b) a nucleic acid sequence partially complementary to an aptamer that becomes available for hybridization to the first guide oligonucleotide after the aptamer binds to the first target analyte; (iv) a second CRISPR complex comprising a Cas13 protein and a second guide oligonucleotide having sufficient complementarity to hybridize to (a) a second target analyte or (b) a nucleic acid sequence partially complementary to an aptamer that becomes available for hybridization to the second guide oligonucleotide after the aptamer binds to the second target analyte. In some embodiments, the guide oligonucleotide is a guide RNA. In various embodiments, the first reporter and/or the second reporter comprises a modified oligonucleotide.

In some embodiments, the kit comprises an additional vessel comprising a substrate for the detectable marker.

In some embodiments, the kit also provides instructions for use. In some embodiments, the kit comprises a swab for acquiring a sample from a subject.

### EXAMPLES

### Example 1

In this example, enzymes were conjugated to a biotinylated oligonucleotide. The resulting enzyme-oligonucleotide conjugates were then attached to a streptavidin-coated surface by simple incubation. A solution containing CRISPR Cas 13 with the guide RNA was then added. In the presence of a target analyte, an RNA sequence activated the CRISPR Cas13. This activated Cas13 cleaved surface-bound enzyme (HRP or horseradish peroxidase) oligonucleotide conjugates which were then released into solution. The solution is retrieved and treated with equal volume of TMB Ultra (enzyme substrate). In the presence of TMB, a blue color was generated. In various embodiments of the disclosure, the enzyme and enzyme substrate may be varied. This procedure is shown schematically in FIG. 1.

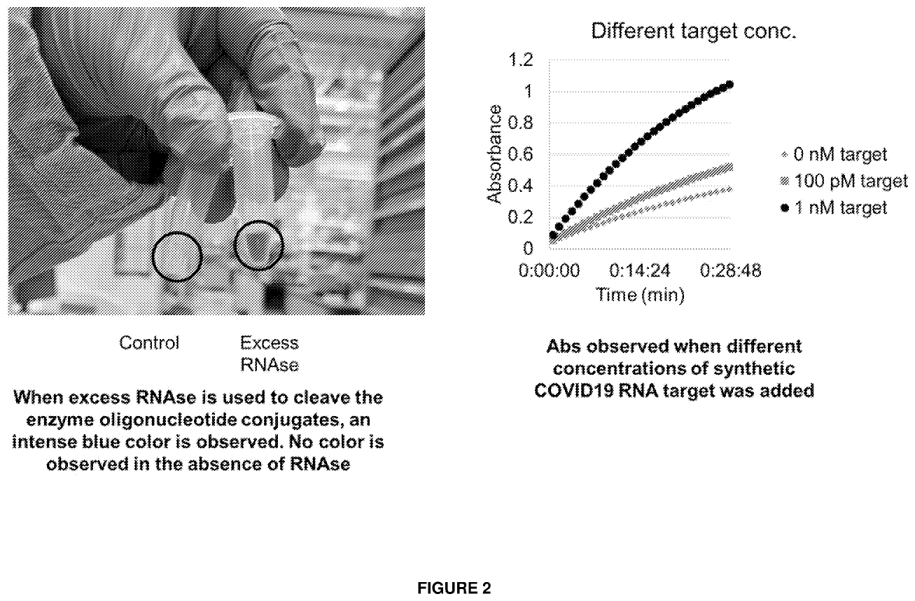

In a typical procedure, 500 nM of biotinylated HRP-RNA conjugate was incubated on to a streptavidin-coated surface for 10 minutes. Next, the surface was washed repeatedly to remove any unattached HRP. Following this washing step, a solution containing Cas13a (100 nM) complexed to a COVID-19 targeting guide RNA (100 nM) was added to the surface. Different concentrations of synthetic COVID-19 RNA targets were added. The addition of target activated Cas13a and resulted in collateral cleavage of the RNA linker between the HRP and the surface. Consequently, HRP was released into the solution. This solution was retrieved and then added to equal volume of TMB Ultra and the absorbance was monitored over time. FIG. 2 shows the results obtained from such an experiment. In the left panel of FIG. 2, results when no RNAse or excess RNAse was added to HRP-based reporters immobilized on magnetic microparticles. In the presence of RNAse alone, the reporters were cleaved and HRP is released into solution. By removing the microbeads and adding TMB to the solution, an intense blue color was observed. No color was observed in the absence of RNAse. In the right panel of FIG. 2, results are shown when increasing amounts of COVID-19 target were added to HRP-based reporters immobilized on magnetic microparticles in the presence of a Cas13-based RNP. As more COVID-19 target was added, more of the reporters were cleaved thereby releasing HRP into solution. Following removal of the microbeads and addition of TMB to the solution, an increase in the amount of absorbance was seen as the concentration of COVID-19 target was increased.

### Example 2

This Example provides additional data generated utilizing methods of the disclosure to generate amplified signal in CRISPR-Cas-based detection. Target recognition activates the CRISPR-Cas complex, leading to catalytic cleavage of oligonucleotide-conjugated horseradish peroxidase (HRP) from the surface of microbeads. This Example shows that the cleaved HRP can be monitored through colorimetric, fluorometric, or luminescent approaches, yielding up to approximately 75-fold turn-on signals and limits of detection as low as approximately 10 fM that enables sensing at clinically relevant concentrations. Importantly, the use of a colorimetric readout allows for rapid (<1 hour), PCR-free, naked eye, room temperature detection of a nucleic acid marker for the SARS-CoV-2 virus. This Example also demonstrates analyte recognition of non-nucleic acid targets. Specifically, ATP binding was interfaced to an aptamer with activation of CRISPR-Cas and subsequent formation of colorimetric signal, enabling the study of ATP in human serum samples.

Nucleic acid-based probes have revolutionized clinical diagnostics due to their ability to sensitively and selectively detect disease biomarkers.^{[1]} Techniques employing polymerase chain reaction (PCR) that can amplify low quantities of nucleic acid targets constitute the gold standard,^{[2]} offering sensitivity as low as one copy per microliter in patient samples.^{[3]} However, target amplification is only possible for nucleic acids, limiting the scope of analytes that can be measured with these assays. Furthermore, PCR is generally not translatable as a method for rapid, point-of-care detection.^{[4]} The SARS-CoV-2 pandemic, in particular, has illustrated the urgent need for developing sensing platforms that are not only sensitive but also rapid, reliable, and deployable in low-resource settings.

Several nucleic acid-based strategies have been developed towards achieving these capabilities, such as the Verigene platform.^{[5]} Amongst these, CRISPR-Cas mediated detection has recently emerged as a powerful strategy for amplified sensing of targets.^{[6]} CRISPR-based diagnostics leverage enzymes from CRISPR-Cas systems (i.e., Cas12 and Cas13), which exhibit nonspecific endonuclease activity after hybridization of a target, or “initiator”, nucleic acid to the guide RNA (g RNA) of the Cas.

When a short fluorophore-quencher labeled nucleic acid is used as a reporter, the active Cas enzyme degrades this sequence, separating the fluorophore and quencher to yield fluorescence turn-on. Notably, the binding of nucleic acid aptamers to non-nucleic acid targets can also be exploited to activate Cas activity, expanding detection to analytes such as ions and small molecules.^{[7]} CRISPR-Cas diagnostics offer several advantages over PCR, including the lack of need for intricate laboratory setups or thermocycling, relatively fast assay times, and robust selectivity for targets with single nucleotide mismatches.^{[8]} Importantly, these tests retain sensitivity in complex biological media.^{[9]} This has fueled the use of CRISPR diagnostics for a variety of applications, with the most advanced being assays with emergency use authorization for SARS-CoV-2 testing.^{[10]} Although CRISPR-Cas based tests have pushed new frontiers in detection, they also suffer from limitations that make their translation into point-of-care diagnostics challenging. For example, sensing with sufficiently low limits of detection (e.g. SARS-CoV-2 RNA at <100,000 copies/mL^{[11]}, Zika viral RNA at <500 copies/mL^{[12]}, etc.) can still require target amplification that entails multiple procedural steps and high incubation temperatures (55-65° C.).^{[13]} With this in mind, the present disclosure provides a detection platform that is translatable to low-resource settings and generalizable to multiple targets, while maintaining assay sensitivity and accuracy. The methods of the disclosure provide at least the following advantages: (1) simple readout without needing sophisticated instrumentation, (2) reasonable assay time (e.g., <2 hours), (3) minimal steps, (4) room temperature measurement, and (5) reliable detection at relevant concentrations for the target of interest.

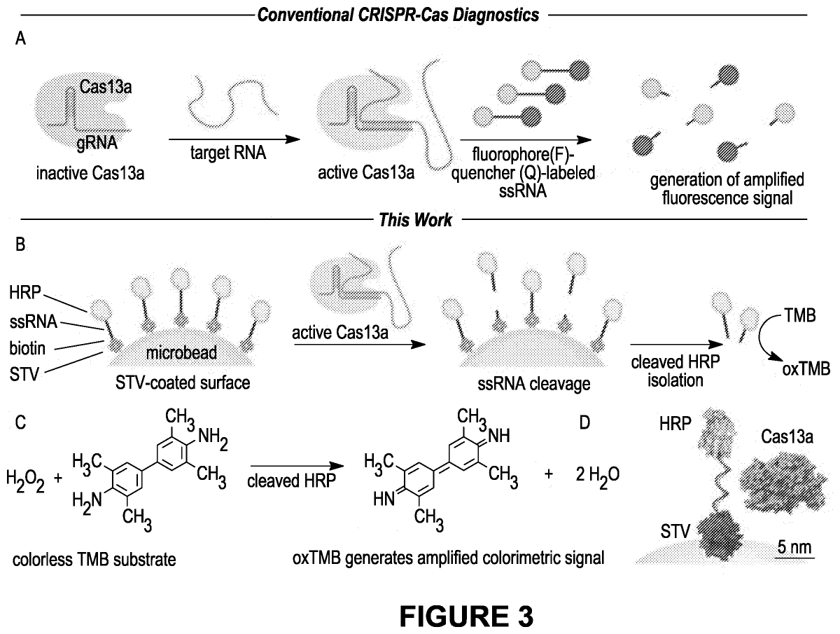

This Example demonstrates a PCR-free CRISPR-mediated platform to enable naked eye detection of both nucleic acid and non-nucleic acid targets. It was hypothesized that a dual enzyme amplification system designed with a Cas enzyme (Cas12a or Cas13a) and horseradish peroxidase (HRP) would generate a robust signal for sensitive detection. HRP was chosen as the enzymatic reporter owing to its ubiquitous use in a variety of commercial assay formats and ability to be detected with high sensitivity via several different signaling substrates.^{[14]} In this strategy (FIG. 3), the Cas enzyme is pre-complexed with a guide RNA (gRNA) to form a ribonucleoprotein complex (RNP). In the presence of the target molecule, a complementary sequence binds to the gRNA and activates the Cas enzyme which then exhibits collateral, non-specific endonuclease activity towards single-stranded oligonucleotides (ssRNA and ssDNA for Cas13 and Cas12, respectively). Consequently, HRP-labeled, surface-bound single stranded oligonucleotides can be rapidly degraded by the active Cas enzyme, thereby liberating free HRP into solution. The free HRP in solution can be detected via colorimetry, fluorescence, or chemiluminescence using appropriate substrates (e.g., 3,3′,5,5′-tetramethylbenzidine for colorimetry, 10-acetyl-3,7-dihydroxyphenoxazine for fluorescence, etc.).

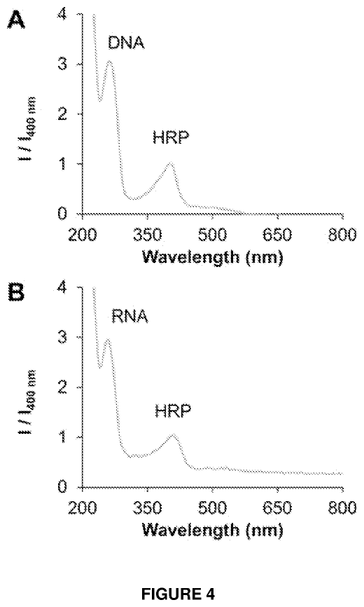

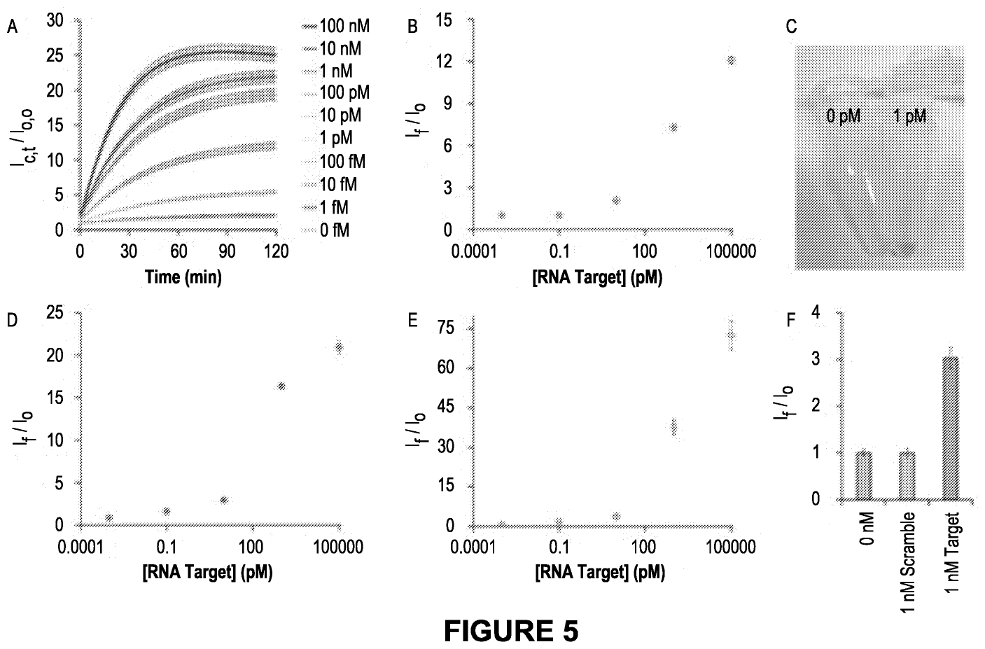

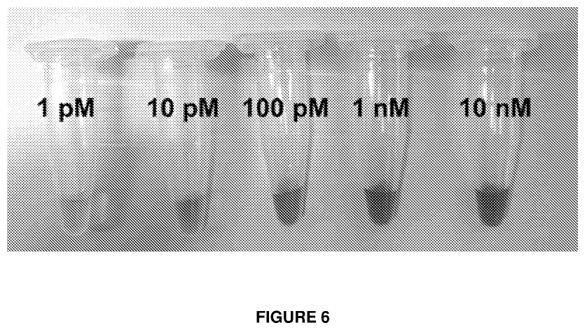

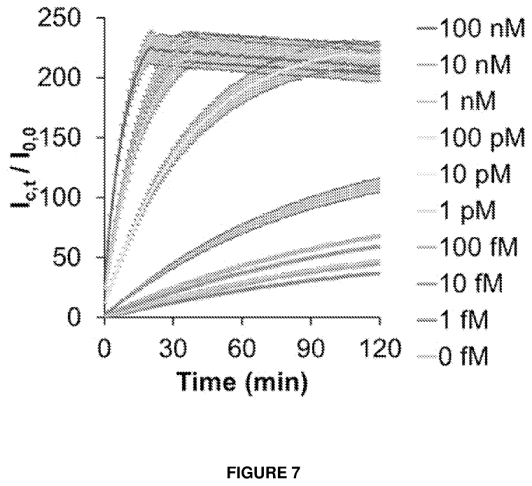

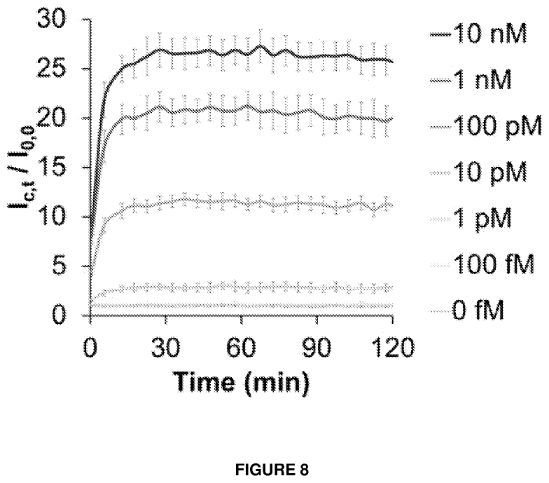

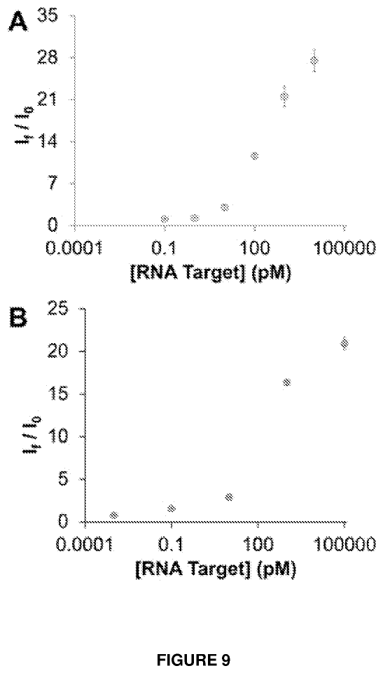

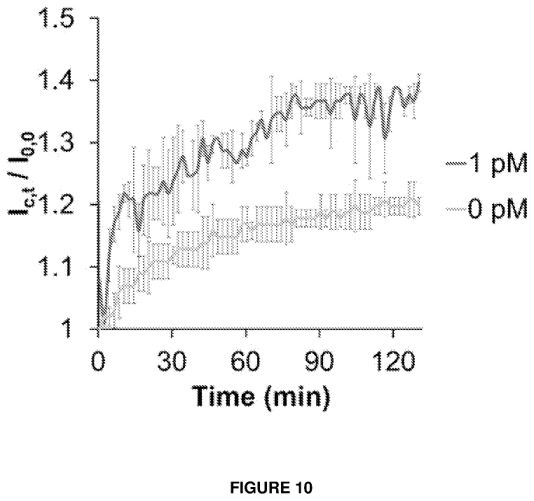

To assess the feasibility of this strategy, a short synthetic transcript corresponding to the ORF1ab gene of the SARS-CoV-2 wildtype virus was used as a model target (Table 1). A 5′-DBCO-U₂₅-biotin-3′ sequence was synthesized and conjugated to azide-labeled HRP using copper-free click chemistry (FIG. 4). The HRP-labeled reporter strands were immobilized on to streptavidin-coated beads and the unbound strands were removed. These beads were then added to a solution containing 12.5 nM of RNP that can bind the target. After 35 minutes of incubation, the beads were separated from the solution via centrifugation and a solution containing the chromogeneic tetramethyl benzidine (TMB) substrate of HRP was added at a 1:1 ratio (v/v). The absorbance of the solution was monitored over time. A blue color developed gradually, with solutions at higher target concentrations exhibiting more intense color. At each concentration, the ratio I_{c,t}/I_{0,0} was calculated, where I_{c,t} is the absorbance at time, t, when a concentration, c, of the target is added and I_{0,0} is the signal intensity at the initial timepoint in the absence of the target (FIG. 5A). The enhancement factor, defined as the absorbance obtained in the presence of the target (I_{c,t}≡I_{f}) relative to the absorbance obtained without the target (I_{0,t}≡I₀), was also calculated. The enhancement factor increased with higher target concentrations and longer incubation times, saturating at approximately 15-fold. From the calibration curve (FIG. 5B), the limit of detection (LOD) was calculated to be approximately 400 fM for colorimetric readouts and 1 pM could be detected visually (FIG. 5C and FIG. 6). The LOD improved to approximately 10 fM when fluorogenic (FIG. 5D and FIG. 7) or luminogenic (FIG. 5E) HRP substrates are used which is approximately 30-fold better than that obtained when a single amplification step with Cas13 and fluorophore-quencher reporters is used (FIG. 8 and FIG. 9). The LOD afforded by this assay approaches the acceptable LOD (approximately 2 fM) outlined by the World Health Organization (WHO) for detecting a viral load for likely disease transmission.^{[11]} This platform is also selective; when a non-complementary RNA target was used, a negligible change in enhancement factor was observed (FIG. 5F). Importantly, approximately 700 fM of the full length transcript of SARS-CoV-2 in solution could be detected colorimetrically (FIG. 10).

TABLE 1
Oligonucleotide sequences used in this study

| | Sequence | SEQ |
|---|---|---|
| | (from 5′ end | ID |
| Identifier | to 3′ end) | NO |
| HRP-RNA | DBCO TEG-UUU UUU UUU | 3 |
| | UUU UUU UUU UUU | |
| | UUU U- biotin | |
| HRP-DNA | DBCO TEG-TTT TTT TTT | 4 |
| | TTT TTT TTT TTT TTT T- | |
| | biotin | |
| ORF1ab gRNA | GAU UUA GAC UAC CCC | 5 |
| | AAA AAC GAA GGG | |
| | GAC UAA AAC CCA ACC | |
| | UCU UCU GUA AUU | |
| | UUU AAA CUA U | |
| ORF1ab RNA | AUA GUU UAA AAA UUA | 6 |
| target | CAG AAG AGG UUG G | |
| Scramble RNA | CUU CUU CAG GUU GGA | 7 |
| | CAG CUG GUG CUG C | |
| ATP gRNA | UAA UUU CUA CUA AGU | 8 |
| | GUA GAU AAG GUU | |
| | UGU GUG UUU ACC UG | |
| ATP Aptamer 1 | ACC TGG GGG AGT ATT | 9 |
| | GCG GAG GAA GGT | |
| | TTG TGT | |
| ATP Aptamer 2 | GTT TAC CTG GGG GAG | 10 |
| | TAT TGC GGA GGA | |
| | AGG T | |
| ATP Initiator | CCC AGG TAA ACA CAC | 11 |
| DNA | AAA CCT T | |

Because target amplification (via, e.g., PCR) is not feasible with non-nucleic acid targets, a highly sensitive signal amplification scheme would be particularly advantageous for these analytes. In this regard, expansion of the scope of recognition of the dual-amplification methods disclosed herein to the detection of non-nucleic acid targets was next tested.

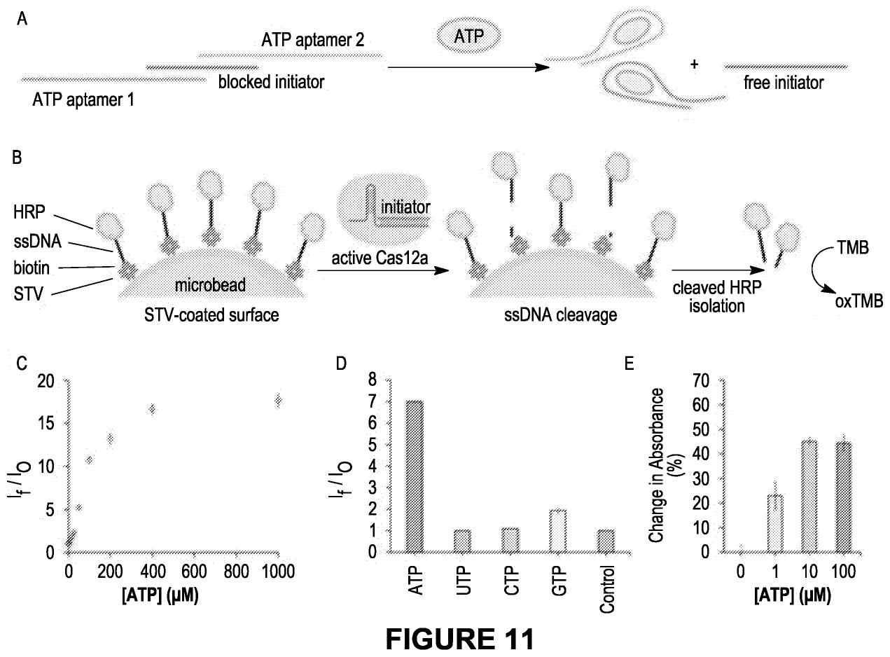

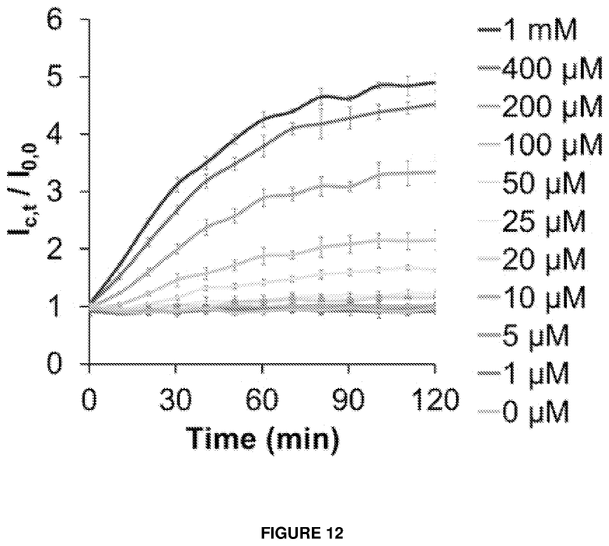

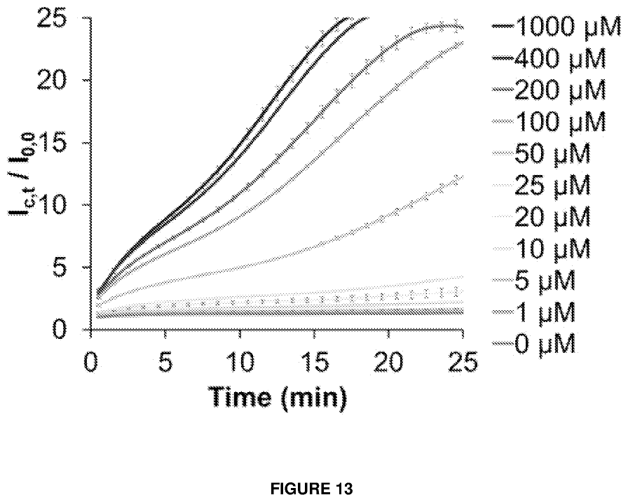

Recently, Lu et al. have reported that Cas-based signaling can be activated by non-nucleic acid targets via aptamer-mediated recognition.^{[7]} As a model target, ATP, which has a well-known DNA aptamer, was chosen. An initiator sequence that can bind to a complementary gRNA and activate Cas12 was used (Table 1). The initiator was first hybridized to two ATP-binding aptamer sequences as shown in FIG. 11A) to prohibit binding to the gRNA. In the presence of ATP, aptamer-ATP complex formation resulted in the generation of the free initiator which then activated the Cas12/gRNA RNP (FIG. 11B and FIG. 12), leading to the cleavage of surface-bound, HRP-labeled T₂₅ sequences. For ATP detection, the aptamer-initiator complex was first incubated with varying concentrations of ATP for 35 minutes, before proceeding with the Cas-based colorimetric assay (FIG. 13). Using this procedure, a maximum enhancement of 25-fold was observed and the LOD was calculated to be approximately 0.2 μM (FIG. 11C). Significantly, this LOD surpassed that of several commercially available colorimetric ATP detection kits.^{[20,21]} This assay is selective for ATP as structurally similar nucleoside triphosphate molecules such as GTP, CTP, and UTP did not elicit significant signal enhancements (FIG. 11D).

To investigate the potential of this platform to detect analytes in complex biological media, known concentrations of ATP were spiked into human serum (1 μM, 10 μM, and 100 μM). The human serum was then diluted 10-fold and subjected to the assay. The data showed that 1 μM ATP in human serum colorimetrically was clearly detected, without interference from the molecules present in serum (FIG. 11E).

In conclusion, this Example showed the efficacy of the dual amplification sensing methods as described herein that couples analyte induced Cas-activation to subsequent release of a detectable marker (e.g., HRP) into solution. Importantly, in the case of nucleic acid targets, this scheme obviates the need for PCR and enabled room temperature analyte sensing with a LOD as low as approximately 10 fM. The ability to couple detectable marker (e.g., HRP) measurement with a variety of signal transduction methods bodes well for this strategy's use in a range of applications. Here, this capability made possible the sensitive, naked eye colorimetric detection of a nucleic acid sequence for the SARS-CoV-2 virus. In addition, this versatility allowed for transducing signal with a fluorescence-based readout, leading to an approximate 30-fold improvement in LOD compared to conventional fluorophore/quencher Cas-based detection in the absence of PCR. Notably, by using an aptamer and blocking strand in the design, the scope of recognition was expanded to non-nucleic acid targets. This gave rise to a probe set that could colorimetrically sense ATP down to 1 μM in human serum samples. The dual amplification strategies described herein are advantageously useful for non-nucleic acid targets considering that PCR is not possible for these analytes. Taken together, the ability to detect a large range of targets across a wide breadth of signaling methods lends the dual amplification strategies of the disclosure well to being a versatile sensing approach for facile point-of-care diagnosis or highly sensitive sample analysis in centralized facilities.

### Additional Results and Discussion

Characterization of HRP-labeled oligonucleotides. The HRP-labelled RNA and DNA conjugates were characterized with UV-Vis spectroscopy. Extinction coefficients for HRP (ε₄₀₀=102,000 M⁻¹ cm⁻¹), DNA (ε₂₆₀=211,100 M⁻¹ cm⁻¹), and RNA (ε₂₆₀=181,200 M⁻¹ cm⁻¹) were used to calculate concentrations of each species. The ratio of oligonucleotide concentration to HRP concentration was used to calculate degree of loading.

Detection of short synthetic SARS-CoV-2 transcript using CRISPR-Cas13 and fluorophore-quencher pairs. A conventional fluorophore-quencher strategy was used (described below) to assess the ability of the CRISPR-Cas13 complex to detect a short synthetic SARS-CoV-2 target. The change in fluorescence enhancement over time is shown in FIG. 8.

The fluorescence turn-on in the presence of target with either a conventional fluorophore-quencher strategy (FIG. 9A) or using the strategy reported herein with a fluorogenic HRP substrate (FIG. 9B) was also compared. This showed that the fluorogenic HRP substrate leads to an approximately 30-fold improvement in sensitivity compared to the fluorophore-quencher reporter.

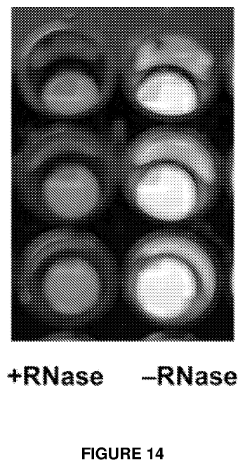

To assess activity of cleaved HRP-RNA conjugates, HRP-RNA beads were incubated with RNase A for 10 minutes. The tubes were then centrifuged (20 k rcf for 2 minutes) to separate the cleaved HRP-RNA conjugates from the beads. 40 μL of the supernatant each sample was transferred to a 96-well plate and 40 μL TMB substrate was added to each well. The positive RNase A control samples exhibited a bright blue visual signal, while samples without RNase A remained clear (FIG. 14).

Colorimetric detection of short synthetic SARS-CoV-2 transcript. Samples with varying ORF1ab RNA target concentrations were visually monitored after addition of TMB substrate to ascertain whether color change could be readily differentiated.

Fluorescence detection of short synthetic SARS-CoV-2 transcript. A calibration curve was constructed to assess the ability to detect a short synthetic SARS-CoV-2 target using the dual signal-amplification method described herein interfaced with a fluorogenic substrate for HRP. The fluorescence signal generation in the presence of varying concentrations of target was monitored over time. High concentrations of target lead to rapid substrate conversion, indicated by high I_{c,t}/I_{0,0} values. At sufficiently long timepoints, a decrease in signal was observed owing to precipitation of substrate from solution. See FIG. 7.

Colorimetric detection of the full SARS-CoV-2 transcript. To assess the ability to detect a fragment of a long and complex RNA sequence, the dual amplification colorimetric method as described herein was applied to a full wild-type SARS-CoV-2 transcript target (a 29 kb sequence). An approximate 1.13-fold signal enhancement for approximately 700 fM target at 35 minutes was observed. See FIG. 10.

Detection of ATP using CRISPR-Cas12 and fluorophore-quencher pairs. The fluorescence signal generation from fluorophore-quencher reporter DNA in the presence of varying concentrations of short synthetic RNA target was monitored over time. The maximum fluorescence enhancement observed was approximately 5-fold for a 1 mM target. See FIG. 12.

Colorimetric detection of ATP. A calibration curve was constructed to assess the ability to detect ATP with a dual amplification method with a colorimetric substrate for HRP. The colorimetric signal generation in the presence of varying concentrations of target was monitored over time. High concentrations of target lead to rapid substrate conversion, indicated by high I_{c,t}/I_{0,0} values. At sufficiently long timepoints, a decrease in signal was observed owing to precipitation of substrate from solution. See FIG. 13.

### Materials and Methods

### Oligonucleotide Design, Synthesis, Purification, and Characterization

Design. In order to facilitate HRP cleavage from microbead surfaces, oligonucleotide strand tethers were designed to provide access for Cas12a and Cas13a enzymes. HRP-labelled sequences were synthesized by incorporating a dibenzocyclooctyl (DBCO) TEG phosphoramidite to the 5′ end of the nucleic acids and reacting them with azide-modified HRP. To attach HRP-labelled oligonucleotides to a surface the strong binding interaction between streptavidin (STV) and biotin was exploited. A 3′ biotin controlled pore glass bead was utilized to synthesize biotin-labelled nucleic acid sequences to attach to STV-coated microbeads. X-ray crystallography data was used to approximate the size of the protein components used for this assay. It was determined that STV is approximately 5 nm×5 nm×6 nm [X. Fan, J. Wang, X. Zhang, Z. Yang, J.-C. Zhang, L. Zhao, H.-L. Peng, J. Lei, H.-W. Wang, Nature communications 2019, 10, 1-11], HRP is 4 nm×5 nm×6 nm [G. I. Berglund, G. H. Carlsson, A. T. Smith, H. Szöke, A. Henriksen, J. Hajdu, Nature 2002, 417, 463-468], and Cas13a is 11 nm×7 nm×8 nm [A. J. Meeske, N. Jia, A. K. Cassel, A. Kozlova, J. Liao, M. Wiedmann, D. J. Patel, L. A. Marraffini, Science 2020, 369, 54-59]. From these dimensions, it was determined that a 25 nucleotide sequence (U25 for RNA and T25 for DNA) would provide enough spacing between the STV and HRP proteins for Cas enzymes to enter the site and cleave the ssRNA or ssDNA.

To activate the Cas13a enzyme, a gRNA sequence that is complementary to a region of the ORF1ab gene of SARS-CoV-2, reported by Zheng and coworkers, was used [F. Zhang, O. O. Abudayyeh, J. S. Gootenberg, A protocol for detection of COVID-19 using CRISPR diagnostics 2020, 8]. To activate the Cas12a enzyme, a gRNA and ATP initiator sequence DNA reported by Lu and coworkers was used [Y. Xiong, J. Zhang, Z. Yang, Q. Mou, Y. Ma, Y. Xiong, Y. Lu, Journal of the American Chemical Society 2020]. For the ATP detection assay, 2 DNA aptamers that bind to ATP were hybridized to the ATP initiator sequence, blocking its ability to activate Cas12a. Upon binding of ATP to the aptamer sequences, the initiator sequence is freed and able to activate Cas12a.

Synthesis, purification, and characterization. Reagents and supplies used for the solid-phase synthesis of the nucleic acids in this study were purchased from Glen Research. DNA was made using a MerMade12 (MM12, BioAutomation Inc., Plano, Texas, USA) synthesizer. Following synthesis, DNA was detached from the controlled pore glass beads via addition of a 30% ammonium hydroxide solution and subsequent incubation at room temperature for 16 hours. The ammonium hydroxide was then evaporated using an Organomation® Multivap® Nitrogen Evaporator. The samples (in water) were then loaded on a reverse phase high-performance liquid chromatography column (C18 column, Varian ProStar 210, Agilent Technologies Inc., Palo Alto, CA, USA) and run using a 0-75% ramp of acetonitrile over 45 min (A=triethylammonium acetate buffer). Following lyophilization of the product fraction, the 4,4′-dimethoxytrityl protecting group was detached via addition of a 20% acetic acid solution and subsequent incubation for 1 hour at room temperature. The protection group was separated out of solution using an ethyl acetate extraction. The DNA in acetic acid was then lyophilized. After this step, the samples were reconstituted in water and characterized via Matrix-assisted laser desorption ionization time-of-flight mass spectrometry (MALDI-TOF MS) and concentration was measured using UV-vis spectroscopy.

RNA was synthesized with 2′-O-triisopropylsilyloxymethyl-protected phosphoramidites (ChemGenes) using a MerMade12 synthesizer (MM12, Bioutomation Inc., Plano, Texas, USA). Following synthesis, cleavage from controlled pore glass beads, and purification via RP-HPLC, RNA strands were deprotected in a triethylamine trihydrofluoride solution for 2 hours at 55° C. A tris buffer was then added to the strands to quench the reaction and the samples were then run through a NAP25 desalting column and lyophilized. After this step, the RNA samples were reconstituted in water and characterized via Matrix-assisted laser desorption.

The ORF1ab gRNA sequence (Table 1) was purchased from Integrated DNA Technologies.

### Synthesis of HRP-Labeled Oligonucleotides

HRP-RNA conjugates. To synthesize HRP-labeled oligonucleotides, 2 mg of HRP (ThermoFisher Scientific Item No. 31490) were dissolved in 1000 μL of 0.1 M NaHCO3 to yield a 50 μM HRP solution. Next, approximately 1000 fold molar excess of Azido-PEG4-NHS ester (ThermoFisher Scientific Item No. 26130) linkers were introduced to the solution and allowed to react with the lysine residues on the HRP surface for 2 hours at room temperature. The azide-functionalized HRP was then passed through a NAP10 desalting column to remove excess linker. The azide-functionalized HRP solution was then concentrated by passing through 30 kDa MWCO spin filters (centrifuged at 4000 rcf for five minutes, three times). Next, azide-functionalized HRP was functionalized with 5′-DBCO TEG-U25-biotin-3′ RNA sequences. For this reaction, 200 μL of 10 μM azide-functionalized HRP and 10 equiv. of RNA were shaken for 15 hours in RNAse-free PBS solution at room temperature. The HRP-RNA conjugates were washed twice with 30 kDa MWCO spin filters (centrifuged at 4000 rcf for five minutes).

HRP-DNA conjugates. To synthesize HRP-labeled DNA, 2 mg of HRP were dissolved in 1000 μL of 0.1 M NaHCO3 to yield a 50 μM HRP solution. Next, approximately 1000 fold molar excess of Azido-PEG4-NHS ester linkers were introduced to the solution and allowed to react with the lysine residues on the HRP surface for 2 hours at room temperature. The azide-functionalized HRP was then passed through a NAP10 desalting column to remove excess linker. The azide-functionalized HRP solution was then concentrated by passing through 50 mL 30 kDa MWCO spin filters (centrifuged at 4000 rcf for five minutes, three times). Next, azide-functionalized HRP was functionalized with 5′-DBCO TEG-T25-biotin-3′ DNA sequences. For this reaction, 200 μL of 10 μM azide-functionalized HRP and 2 equiv. of DNA were shaken for 15 hours in DNAse-free PBS solution at room temperature. The HRP-DNA conjugates were washed twice with 30 kDa MWCO spin filters (centrifuged at 4000 rcf for five minutes).

### Immobilization of HRP-Labeled Oligonucleotides onto Streptavidin-Coated Beads

HRP-RNA beads. Microbead surfaces were functionalized with HRP-RNA conjugates. First, 20 μL of streptavidin-coated beads (Sigma-Aldrich, Item No. 08014) were added to 600 μL of RNase-free PBS containing 0.1% Tween 20. Next, 2.5 μL of 800 μM HRP-RNA-biotin was introduced and the solution was shaken at 1500 rpm for 5 minutes. To separate the unreacted HRP-RNA-biotin, the solution was centrifuged for 1 minute at 20 k rcf, such that the beads were pelleted at the bottom of the tube and the supernatant could be removed. The beads were subsequently washed eight times with 1× PBS containing 0.1% Tween 20.

HRP-DNA beads. Microbead surfaces were functionalized with HRP-DNA conjugates. First, 40 μL of streptavidin-coated beads were added to 600 μL of RNase-free PBS containing 0.1% Tween 20. Next, 5 μL of 800 μM of HRP-RNA-biotin was introduced and the solution was shaken at 1500 rpm for 5 minutes. To separate the unreacted HRP-RNA-biotin, the solution was centrifuged for 1 minute at 20 k rcf, such that the beads were pelleted at the bottom of the tube and the supernatant could be removed. The beads were subsequently washed eight times with 1× PBS containing 0.1% Tween 20.

### Detection Assays

All experiments were performed in triplicate unless otherwise stated.

Detection of short synthetic SARS-CoV-2 transcript using CRISPR-Cas13 and a fluorophore-quencher pair. To assess the limit of detection using a fluorophore-quencher RNA reporter, a 1 mL solution of 25 nM Cas13a (MCLAB Item No. CAS13a-100), 25 nM ORF1ab gRNA and 400 nM RNaseAlert substrate (ThermoFisher Scientific Item No. AM1964) was prepared in Buffer 1 (20 mM Hepes, 50 mM KCl, 5 mM MgCl₂). 50 μL solutions of ORF1ab RNA target of varying concentrations (20 nM, 2 nM, 200 pM, 20 pM, 2 pM, 200 fM, 0 fM) were prepared in Buffer 1 and combined with 50 μL of the Cas13a-gRNA containing solution such that the final RNA target concentrations were 10 nM, 1 nM, 100 pM, 10 pM, 1 pM, 100 fM, and 0 fM. A fluorescence reading was taken on a BioTek Cytation 5 plate reader (excitation 480 nm, emission 520 nm) at 5 minute intervals over a 2 hour period.

Colorimetric detection of short synthetic SARS-CoV-2 transcript. A calibration curve of the dual amplification colorimetric sensing of RNA reported herein was prepared. First, 6 tubes of HRP-RNA microbeads were prepared as reported above and combined into one tube. Next, a 1 mL solution of 12.5 nM Cas13a and 12.5 nM ORF1ab gRNA was prepared in Buffer 1 and added to the HRP-RNA beads. 50 μL aliquots of this solution were then prepared in tubes. 1.5 μL solutions of ORF1ab RNA target of varying concentrations was added to each tube yielding final target concentrations of 100 nM, 10 nM, 1 nM, 100 pM, 10 pM, 1 pM, 100 fM, 10 fM, 1 fM, 0 fM. The tubes were shaken for 35 minutes at 1500 rpm. The tubes were then centrifuged (20 k rcf for 2 minutes) to separate the cleaved HRP-RNA conjugates from the beads. 40 μL of the supernatant each sample was transferred to a 96-well plate and 40 μL TMB substrate was added to each well. An absorbance reading was taken on a BioTek Cytation 5 plate reader (650 nm) at 1 minute intervals over a 2 hour period.

Fluorescence detection of short synthetic SARS-CoV-2 transcript. A calibration curve of the dual amplification fluorometric sensing of RNA reported herein was prepared. First, 6 tubes of HRP-RNA microbeads were prepared as reported above and combined into one tube. Next, a 1 mL solution of 12.5 nM Cas13a and 12.5 nM ORF1ab gRNA was prepared in Buffer 1 and added to the HRP-RNA beads. 50 μL aliquots of this solution were then prepared in tubes. 1.5 μL solutions of ORF1ab RNA target of varying concentrations was added to each tube yielding final target concentrations of 100 nM, 10 nM, 1 nM, 100 pM, 10 pM, 1 pM, 100 fM, 10 fM, 1 fM, 0 fM. The tubes were shaken for 35 minutes at 1500 rpm. The tubes were then centrifuged (20 k rcf for 2 minutes) to separate the cleaved HRP-RNA conjugates from the beads. 40 μL of the supernatant each sample was transferred to a 96-well plate and 40 μL 10-Acetyl-3,7-dihydroxyphenoxazine (ADHP) (ThermoFisher Scientific Item No. 15159) was added to each well. A fluorescence reading was taken on a BioTek Cytation 5 plate reader (excitation 570 nm, emission 585 nm) at 2 minute intervals over a 2 hour period.

Luminescence detection of short synthetic SARS-CoV-2 transcript. A calibration curve of the dual amplification luminometric sensing of RNA method reported herein was prepared. First, 6 tubes of HRP-RNA microbeads were prepared as reported above and combined into one tube. Next, a 1 mL solution of 12.5 nM Cas13a and 12.5 nM ORF1ab gRNA was prepared in Buffer 1 and added to the HRP-RNA beads. 50 μL aliquots of this solution were then prepared in tubes. 1.5 pL solutions of ORF1ab RNA target of varying concentrations was added to each tube yielding final target concentrations of 100 nM, 10 nM, 1 nM, 100 pM, 10 pM, 1 pM, 100 fM, 10 fM, 1 fM, 0 fM. The tubes were shaken for 35 minutes at 1500 rpm. The tubes were then centrifuged (20 k rcf for 2 minutes) to separate the cleaved HRP-RNA conjugates from the beads. 40 μL of the supernatant each sample was transferred to a 96-well plate and 40 μL of SuperSignal ELISA Femto chemiluminescent substrate (ThermoFisher Scientific Item No. 37074) was added to each well. A luminescence reading was taken on a BioTek Cytation 5 plate reader at 1 minute intervals over a 10 minute period.

Colorimetric detection of the full SARS-CoV-2 transcript. The ability to detect the full RNA transcript of the SARS-CoV-2 virus was assessed. First, 20 μL of 1 million copies/μL of SARS-CoV-2 Synthetic RNA transcript was lyophilized. Next, 2 tubes of HRP-RNA microbeads were prepared as reported above and combined into one tube. A 1 mL solution of 12.5 nM Cas13a and 12.5 nM ORF1ab gRNA was prepared in Buffer 1 and added to the HRP-RNA beads. 50 μL aliquots of this solution were then added to the dried RNA tubes (or empty tubes for the 0 pM control). The tubes were shaken for 90 minutes at 1500 rpm. The tubes were then centrifuged (20,000 rcf, 2 minutes) to separate the cleaved HRP-RNA conjugates from the beads. 40 μL of the supernatant each sample was transferred to a 96-well plate and 40 μL TMB substrate was added to each well. An absorbance reading was taken on a BioTek Cytation 5 plate reader (650 nm) at 1 minute intervals over a 2 hour period.

Specificity test for SARS-CoV-2. Specificity of the dual amplification colorimetric sensing of RNA reported herein was studied. First, 3 tubes of HRP-RNA microbeads were prepared as reported above and combined into one tube. Next, a 1 mL solution of 12.5 nM Cas13a and 12.5 nM ORF1ab gRNA was prepared in Buffer 1 and added to the HRP-RNA beads. 50 μL aliquots of this solution were then prepared in tubes. The tubes were subsequently spiked with 1 nM ORF1ab target RNA, 1 nM of Scramble RNA or 0 nM control. The tubes were shaken for 35 minutes at 1500 rpm. The tubes were then centrifuged (20 k rcf for 2 minutes) to separate the cleaved HRP-RNA conjugates from the beads. 40 μL of the supernatant each sample was transferred to a 96-well plate and 40 μL TMB substrate was added to each well. An absorbance reading was taken on a BioTek Cytation 5 plate reader (650 nm) at 1 minute intervals over a 2 hour period.

Detection of ATP using CRISPR-Cas12 and fluorophore-quencher pairs. To assess the limit of detection of ATP sensing using fluorophore-quencher DNA reporters, a 1 mL solution of 10 nM Cas12a (New England Biolabs Item No. AM1970), 10 nM ATP gRNA and 400 nM DNaseAlert substrate (ThermoFisher Scientific Item No. AM1964) was prepared in Buffer 2 (40 mM Tris, 100 mM NaCl, 20 mM MgCl₂). Next, a solution containing 25 nM of ATP Aptamer 1, 25 nM of ATP Aptamer 2, and 12.5 nM ATP initiator DNA was prepared in Buffer 2. The DNA strands were annealed at 80° C. for 10 minutes and allowed to cool to room temperature. Next, ATP was spiked into 50 μL solutions of the DNA with varying concentrations (1 mM, 400 μM, 200 μM, 100 μM, 50 μM, 25 μM, 20 μM, 10 μM, 5 μM, 1 μM, 0 μM). The solutions were shaken for 35 minutes, to allow aptamer binding to ATP and the release of free initiator DNA. Next, 50 μL of the Cas12a-gRNA containing solution was added to each tube and a fluorescence reading was taken on a BioTek Cytation 5 plate reader (excitation 480 nm, emission 520 nm) at 5 minute intervals over a 2 hour period.

Colorimetric detection of ATP. A calibration curve of the dual amplification colorimetric sensing of ATP reported herein was prepared. First, 4 tubes of HRP-DNA microbeads were prepared as reported above and combined into one tube. Next, a solution containing 25 nM of ATP Aptamer 1, 25 nM of ATP Aptamer 2, and 12.5 nM ATP initiator DNA prepared in Buffer 2. The DNA strands were annealed at 80° C. for 10 minutes and allowed to cool to room temperature. ATP was spiked into 50 μL solutions of the DNA with varying concentrations (1 mM, 400 μM, 200 μM, 100 μM, 50 μM, 25 μM, 20 μM, 10 μM, 5 μM, 1 μM, 0 μM). The solutions were shaken for 35 minutes, to allow aptamer binding to ATP and the release of free initiator DNA. Next, a 1 mL solution of 10 nM Cas12a and 10 nM ATP gRNA was prepared in Buffer 2 and added to the HRP-DNA beads. 50 μL aliquots of this solution were then added to the ATP containing tubes. The tubes were shaken for 35 minutes at 1500 rpm. The tubes were then centrifuged (20 k rcf for 2 minutes) to separate the cleaved HRP-DNA conjugates from the beads. 40 μL of the supernatant each sample was transferred to a 96-well plate and 40 μL TMB substrate (TMB) was added to each well. An absorbance reading was taken on a BioTek Cytation 5 plate reader (650 nm) at 1 minute intervals over a 2 hour period.

Specificity test for ATP. Specificity of the dual amplification colorimetric sensing of ATP reported herein was assessed. First, 4 tubes of HRP-DNA microbeads were prepared as reported above and combined into one tube. Next, a solution containing 25 nM of ATP Aptamer 1, 25 nM of ATP Aptamer 2, and 12.5 nM ATP initiator DNA was prepared in Buffer 2. The DNA strands were annealed at 80° C. for 10 minutes and allowed to cool to room temperature. ATP, or structurally similar nucleoside triphosphate molecules GTP, CTP, or UTP was spiked into 50 μL solutions of the DNA at 100 nM concentrations. The solutions were shaken for 35 minutes, to allow aptamer binding and the release of free initiator DNA. Next, a 1 mL solution of 10 nM Cas12a and 10 nM ATP gRNA was prepared in Buffer 2 and added to the HRP-DNA beads. 50 μL aliquots of this solution were then added to the ATP containing tubes. The tubes were shaken for 35 minutes at 1500 rpm. The tubes were then centrifuged (20 k rcf for 2 minutes) to separate the cleaved HRP-DNA conjugates from the beads. 40 μL of the supernatant each sample was transferred to a 96-well plate and 40 μL TMB substrate was added to each well. An absorbance reading was taken on a BioTek Cytation 5 plate reader (650 nm) at 1 minute intervals over a 2 hour period.

Detection of ATP in serum. Detection of ATP using the dual amplification colorimetric sensing of in human serum was assessed. First, 2 tubes of HRP-DNA microbeads were prepared as reported above and combined into one tube. Next, a solution containing 50 nM of ATP Aptamer 1, 50 nM of ATP Aptamer 2, and 25 nM ATP initiator DNA were prepared in Buffer 2. The DNA strands were annealed at 80° C. for 10 minutes and allowed to cool to room temperature. ATP was spiked into 25 μL solutions of 20% human serum (10 μL serum, 40 μL Buffer 2) with varying concentrations (100 μM, 10 μM, 1 μM, 0 μM). 25 μL of the annealed DNA solution was added to each tube and the tubes were shaken for 35 minutes, to allow aptamer binding to ATP and the release of free initiator DNA. Next, a 1 mL solution of 10 nM Cas12a and 10 nM ATP gRNA was prepared in Buffer 2 and added to the HRP-DNA beads. 50 μL aliquots of this solution were then added to the ATP containing tubes. The tubes were shaken for 35 minutes at 1500 rpm. The tubes were then centrifuged (20 k rcf for 2 minutes) to separate the cleaved HRP-DNA conjugates from the beads. 40 μL of the supernatant each sample was transferred to a 96-well plate and 40 μL TMB substrate was added to each well. An absorbance reading was taken on a BioTek Cytation 5 plate reader (650 nm) at 1 minute intervals over a 2 hour period.

### Data Analysis and Statistics

In all figures, values and error bars represent the mean and standard deviation, respectively, of several independent readings (three readings). To determine the appropriate assay time for each method, signal intensity was monitored at regular intervals. I_{c,t}/I_{0,0} was plotted with respect to time, where I_{c,t} denotes the concentration-dependent signal intensity (absorbance for colorimetric, fluorescence for fluorometric and luminescence for luminometric assays) and I_{0,0} denotes the initial signal in the absence of the target at the start of the experiment. Assay time was determined by the time point at which signal enhancement was significant for low concentration samples. Enhancement factor was calculated using Equation 1.

$$E . F . = \frac{{I}_{f}}{{I}_{0}} \qquad eq ( 1 )$$

where I₀ denotes the initial signal intensity of the cleaved HRP in the absence of the target and I_{f} denotes its final signal upon addition of the target analyte. Absolute percent change in signal was calculated using Equation 2:

$$Δ I ( \% ) = \frac{\vert {I}_{f} - {I}_{0} \vert}{{I}_{0}} \cdot 100 \qquad eq ( 2 )$$

where I₀ denotes the initial signal in the absence of the target and I_{f} denotes its final signal upon addition of the target analyte.

For all analytes, the limit of detection (LOD) was determined by the 3σ/m method, where a denotes the standard deviation of the response and m denotes the initial slope of the calibration curve.

### REFERENCES

[1] a) S. B. Ebrahimi, D. Samanta, C. A. Mirkin, Journal of the American Chemical Society 2020, 142, 11343-11356; b) D. Samanta, S. B. Ebrahimi, C. A. Mirkin, Advanced Materials 2020, 32, 1901743; c) N. L. Rosi, C. A. Mirkin, Chem Rev 2005, 105, 1547-1562; d) D. Samanta, S. B. Ebrahimi, C. A. Mirkin, Adv Mater 2020, 32, e1901743.
[2] R. K. Saiki, S. Scharf, F. Faloona, K. B. Mullis, G. T. Horn, H. A. Erlich, N. Arnheim, Science 1985, 230, 1350-1354.
[3] a) Z. Farka, M. J. Mickert, M. Pastucha, Z. Mikušová, P. Skládal, H. H. Gorris, Angewandte Chemie International Edition 2020, 59, 10746-10773; b) C. B. F. Vogels, A. F. Brito, A. L. Wyllie, J. R. Fauver, I. M. Ott, C. C. Kalinich, M. E. Petrone, A. Casanovas-Massana, M. Catherine Muenker, A. J. Moore, J. Klein, P. Lu, A. Lu-Culligan, X. Jiang, D. J. Kim, E. Kudo, T. Mao, M. Moriyama, J. E. Oh, A. Park, J. Silva, E. Song, T. Takahashi, M. Taura, M. Tokuyama, A. Venkataraman, O.-E. Weizman, P. Wong, Y. Yang, N. R. Cheemarla, E. B. White, S. Lapidus, R. Earnest, B. Geng, P. Vijayakumar, C. Odio, J. Fournier, S. Bermejo, S. Farhadian, C. S. Dela Cruz, A. Iwasaki, A. I. Ko, M. L. Landry, E. F. Foxman, N. D. Grubaugh, Nature Microbiology 2020, 5, 1299-1305.
[4] a) D. B. Larremore, B. Wilder, E. Lester, S. Shehata, J. M. Burke, J. A. Hay, M. Tambe, M. J. Mina, R. Parker, Science advances 2021, 7, eabd5393; b) M. J. Mina, R. Parker, D. B. Larremore, New England Journal of Medicine 2020, 383, e120.
[5] a) T. A. Taton, C. A. Mirkin, R. L. Letsinger, Science 2000, 289, 1757-1760; b) T. Notomi, H. Okayama, H. Masubuchi, T. Yonekawa, K. Watanabe, N. Amino, T. Hase, Nucleic acids research 2000, 28, e63-e63.
[6] a) J. S. Gootenberg, O. O. Abudayyeh, J. W. Lee, P. Essletzbichler, A. J. Dy, J. Joung, V. Verdine, N. Donghia, N. M. Daringer, C. A. Freije, C. Myhrvold, R. P. Bhattacharyya, J. Livny, A. Regev, E. V. Koonin, D. T. Hung, P. C. Sabeti, J. J. Collins, F. Zhang, Science 2017, 356, 438-442; b) J. S. Chen, E. Ma, L. B. Harrington, M. Da Costa, X. Tian, J. M. Palefsky, J. A. Doudna, Science 2018.
[7] Y. Xiong, J. Zhang, Z. Yang, Q. Mou, Y. Ma, Y. Xiong, Y. Lu, Journal of the American Chemical Society 2020.
[8] M. M. Kaminski, O. O. Abudayyeh, J. S. Gootenberg, F. Zhang, J. J. Collins, 2021.
[9] a) H. de Puig, R. A. Lee, D. Najjar, X. Tan, L. R. Soenksen, N. M. Angenent-Mari, N. M. Donghia, N. E. Weckman, A. Ory, C. F. Ng, Science Advances 2021, 7, eabh2944; b) J. Arizti-Sanz, C. A. Freije, A. C. Stanton, B. A. Petros, C. K. Boehm, S. Siddiqui, B. M. Shaw, G. Adams, T.-S. F. Kosoko-Thoroddsen, M. E. Kemball, J. N. Uwanibe, F. V. Ajogbasile, P. E. Eromon, R. Gross, L. Wronka, K. Caviness, L. E. Hensley, N. H. Bergman, B. L. MacInnis, C. T. Happi, J. E. Lemieux, P. C. Sabeti, C. Myhrvold, Nature communications 2020, 11, 5921-5921.
[10] a) G. Guglielmi, Nature 2020; b) P. Kumar, Y. S. Malik, B. Ganesh, S. Rahangdale, S. Saurabh, S. Natesan, A. Srivastava, K. Sharun, M. Yatoo, R. Tiwari, Frontiers in cellular and infection microbiology 2020, 10, 639.
[11] in WHO Target Product Profiles for COVID-19 Diagnostics, Sep. 28, 2020 ed., World Health Organization, Geneva, Switzerland, 2020.
[12] a) F. Moussy, World Health Organization, Geneva, Switzerland, 2016; b) J. S. Gootenberg, O. O. Abudayyeh, M. J. Kellner, J. Joung, J. J. Collins, F. Zhang, Science 2018, 360, 439-444.
[13] a) J. P. Broughton, X. Deng, G. Yu, C. L. Fasching, V. Servellita, J. Singh, X. Miao, J. A. Streithorst, A. Granados, A. Sotomayor-Gonzalez, K. Zorn, A. Gopez, E. Hsu, W. Gu, S. Miller, C. Y. Pan, H. Guevara, D. A. Wadford, J. S. Chen, C. Y. Chiu, Nature Biotechnology 2020; b) J. Joung, A. Ladha, M. Saito, N.-G. Kim, A. E. Woolley, M. Segel, R. P. Barretto, A. Ranu, R. K. Macrae, G. Faure, New England Journal of Medicine 2020, 383, 1492-1494.
[14] D. Calabria, M. M. Calabretta, M. Zangheri, E. Marchegiani, I. Trozzi, M. Guardigli, E. Michelini, F. Di Nardo, L. Anfossi, C. Baggiani, Sensors 2021, 21, 3358.

What is claimed is:

1. A method of detecting a target analyte in a sample, the method comprising:
(A) contacting the sample to a solution comprising:
(i) a reporter comprising an oligonucleotide conjugated to a detectable marker, wherein the reporter is immobilized on a surface;
(ii) a guide oligonucleotide that hybridizes to (a) the target analyte and/or (b) a nucleic acid sequence partially complementary to an aptamer that becomes available for hybridization to the guide oligonucleotide after the aptamer binds to the target analyte; and
(iii) a Cas12 and/or a Cas13 protein that cleaves the reporter after hybridization of the guide oligonucleotide to (a) the target analyte and/or (b) the nucleic acid sequence partially complementary to the aptamer that becomes available for hybridization to the guide oligonucleotide after the aptamer binds to the target analyte, wherein cleavage of the reporter results in release of the detectable marker,
wherein the contacting occurs in a vessel;
(B) removing the solution comprising the released detectable marker from the vessel, and
(C) measuring a signal produced by the released detectable marker in the solution removed from the vessel, wherein the measuring provides for detection of the target analyte in the sample.

2. The method of claim 1, wherein the reporter comprises two or more oligonucleotides conjugated to the detectable marker.

3. The method of claim 1, wherein the reporter consists of one oligonucleotide conjugated to one detectable marker.

4. The method of any one of claims 1-3, wherein the Cas12 protein comprises a sequence as set out in SEQ ID NO: 1.

5. The method of any one of claims 1-4, wherein the Cas13 protein comprises a sequence as set out in SEQ ID NO: 2.

6. The method of any one of claims 1-5, wherein the signal is greater when the target analyte is present in the sample than the signal when the target analyte is not in the sample.

7. The method of any one of claims 1-6, wherein the signal is about 2-fold to 20-fold, 2-fold to 10-fold, 2-fold to 5-fold, 5-fold to 20-fold, or 5-fold to 10-fold greater when the target analyte is present in the sample than the signal when the target analyte is not in the sample.

8. The method of any one of claims 1-6, wherein the signal is about 1.1-fold, 1.2-fold, 1.3-fold, 1.4-fold, 1.5-fold, 1.6-fold, 1.7-fold, 1.8-fold, 1.9-fold, or 2-fold greater when the target analyte is present in the sample than the signal when the target analyte is not in the sample.

9. The method of any one of claims 1-8, wherein the guide oligonucleotide is RNA or a DNA-RNA chimera.

10. The method of any one of claims 1-9, wherein the oligonucleotide portion of the reporter is DNA, RNA, a DNA-RNA chimera, modified forms thereof, or a combination thereof.

11. The method of any one of claims 1-10, wherein the target analyte is a nucleic acid, a protein, a small molecule, an ion, a carbohydrate, a cell, or a combination thereof.

12. The method of claim 11, wherein the ion is a metal ion.

13. The method of claim 12, wherein the metal ion is a mercury ion, a copper ion, a silver ion, a zinc ion, a gold ion, a manganese ion, or a combination thereof.

14. The method of claim 12, wherein the ion is a hydrogen ion.

15. The method of any one of claims 11-13, wherein the nucleic acid is a viral nucleic acid.

16. The method of claim 15, wherein the viral nucleic acid is from a DNA virus, a RNA virus, or a combination thereof.

17. The method of claim 15 or claim 16, wherein the viral nucleic acid is from a Coronaviridae virus, an Arteriviridae virus, a Roniviridae virus, a Picornaviridae virus, or a combination thereof.

18. The method of claim 15 or claim 16, wherein the virus is Coronavirus, MERS, alphacoronavirus HCoV-NL63, betacoronaviruses HCoV-OC43, H1 N1 influenza A, influenza BSARS, or a combination thereof.

19. The method of claim 18, wherein the Coronavirus is SARS-CoV-2 and/or a variant thereof.

20. The method of any one of claims 11-19, wherein the nucleic acid is bacterial nucleic acid.

21. The method of claim 20, wherein the bacterial nucleic acid is from Myobacterium tuberculosis, E. coli, Staphylococcus aureus, Shigella dysenteriae, or a combination thereof.

22. The method of any one of claims 11-21, wherein the nucleic acid is protozoan nucleic acid.

23. The method of claim 22, wherein the protozoan nucleic acid is from Plasmodium falciparum, Plasmodium vivax, Plasmodium ovale, or Plasmodium malariae, or a combination thereof.

24. The method of any one of claims 11-23, wherein the nucleic acid is cancer-related nucleic acid.

25. The method of claim 24, wherein the cancer-related nucleic acid is mRNA, miRNA, circulating DNA, or a combination thereof.

26. The method of claim 24 or claim 25, wherein the cancer-related nucleic acid is BRAF, PIK3CA, MGMT, KRAS, TP53, ESR1, EML4-ALK fusion, miR-125b-5p, miR-155, or a combination thereof.

27. The method of any one of claims 11-26, wherein the protein is prostate-specific antigen (PSA) or thrombin.

28. The method of any one of claims 11-27, wherein the small molecule is adenosine triphosphate (ATP), dehydroepiandrosterone (DHEA), dehydroepiandrosterone sulfate (DHEA-S), or a combination thereof.

29. The method of any one of claims 1-28, wherein the oligonucleotide portion of the reporter is about 2 to about 50 nucleotides in length.

30. The method of any one of claims 1-29, wherein the guide oligonucleotide is about 10 to about 100 nucleotides in length.

31. The method of any one of claim 1-30, wherein the detectable marker is an enzyme or a catalyst.

32. The method of any one of claims 1-31, wherein the surface is a tube, a bead, a multiwell plate, a hydrogel, or a nanoparticle.

33. The method of claim 32, wherein the nanoparticle is magnetic.

34. The method of any one of claims 1-33, wherein the vessel is a tube, or a multiwell plate.

35. The method of any one of claims 1-34, wherein the method is performed at room temperature.
